(19) United States
(12) Patent Application Publication
(10) Pub. No.: US 2025/0262197 A1
(43) Pub. Date: Aug. 21, 2025

(54) COMPOUNDS AND METHODS FOR TREATING CHEMOTHERAPY-INDUCED PAIN

(71) Applicant: LEXICON PHARMACEUTICALS, INC., The Woodlands, TX (US)

(72) Inventors: Suma Gopinathan, The Woodlands, TX (US); Alan George Ernest Wilson, The Woodlands, TX (US)

(73) Assignee: LEXICON PHARMACEUTICALS, INC., The Woodlands, TX (US)

(21) Appl. No.: 19/053,009

(22) Filed: Feb. 13, 2025

Related U.S. Application Data
(60) Provisional application No. 63/553,739, filed on Feb. 15, 2024.

(51) Int. Cl. A61K 31/444; A61P 25/02

(52) U.S. Cl. CPC A61K 31/444; A61P 25/02

(57) ABSTRACT

Disclosed herein are methods and compositions for treating, managing, and preventing chemotherapy-induced pain in a patient in need thereof. Particular methods and compositions utilize an adaptor associated kinase 1 inhibitor of Formula I:

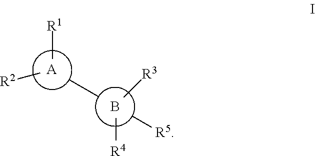

This application claims priority to U.S. provisional patent application No. 63/553,739, filed Feb. 15, 2024, the entirety of which is incorporated herein by reference.

### BACKGROUND

Chemotherapy-induced peripheral neuropathy (CIPN) is a frequent adverse effect of anticancer agents such as platinum, taxanes, proteosome inhibitors, vinca alkaloids, and immunomodulatory drugs. It commonly presents with sensory symptoms in a symmetrical “glove and stocking” distribution: patients typically experience pain, tingling, and numbness beginning at the fingers and toes that progress proximally to involve the arms and legs, which some describe as feeling like they are wearing stockings and gloves when they are not. Desforges, A. D., et al., “Treatment and diagnosis of chemotherapy-induced peripheral neuropathy: An update” Biomed Pharmacother. 2022;147:112671. CIPN typically develops shortly after the onset of, and continues through, chemotherapy treatment. Id.

CIPN is common. It is estimated that approximately 30-40% of patients receiving neurotoxic chemotherapy will suffer from this condition. Staff, N. P., et al., “Chemotherapy-induced Peripheral Neuropathy: A Current Review” Ann Neurol. 2017;81(6):772-781. In patients receiving oxaliplatin, the prevalence of the condition is approximately 68% after one month of chemotherapy, reducing to 60% at three months, and 30% at six months or more. Yang, Y., et al., “Targeting strategies for oxaliplatin-induced peripheral neuropathy: clinical syndrome, molecular basis, and drug development” J Exp Clin Cancer Res 2021;40:331.

Development of CIPN may indicate the need for reducing or discontinuing the use of a chemotherapy agent, but because discontinuation may hamper treatment, oncologists are frequently forced to weigh the risks of quality of life impairments from CIPN and the benefits of possible cancer remission or cure. Staff, supra at 3. CIPN is the most common side effect associated with the dose limiting toxicity of oxaliplatin. Yang, supra at 2.

Oxaliplatin is a form of chelated platinum that is used to manage and treat metastatic colorectal cancer. Devanabanda B, Kasi A. “Oxaliplatin” [Updated 2023 May 16]. In: StatPearls [Internet]. Treasure Island (FL): StatPearls Publishing; 2024 January-. While approved in the United States as an adjunctive treatment of stage III colorectal cancer, it has multiple off-label uses. Id. Acute CIPN, which can begin within hours of the drug's infusion and last for at least the following week, occurs in as many as 96% of patients on oxaliplatin. Yang, supra at 3. Characterized by cold-sensitive peripheral paresthesia and motor symptoms, the degree of acute neuropathy may predict the development of chronic CIPN. Id. Chronic neuropathic pain associated with oxaliplatin—which may present as sensation loss and changes in proprioception that affect daily activities—can last for years. Approximately 60% of patients who took oxaliplatin “report long-lasting neuropathic symptoms that significantly impair their quality of life”. Id.

The etiology of CIPN is complex and involves multiple molecular and physiological mechanisms. There are currently no approved or effective agents for its prevention. Desforges, supra at 1. Duloxetine is the only drug “moderately recommended” by the American Society of Clinical Oncology for the prevention or treatment of CIPN. Yang, supra at 10. The efficacies of other drugs have been tested, but “their prevention of oxaliplatin-induced CIPN is still controversial”. Id.

### SUMMARY

This invention is directed to methods of treating, managing, and preventing chemotherapy-induced peripheral neuropathy (CIPN), which comprise inhibiting adaptor associated kinase 1 (AAK1) in a patient in need thereof. A preferred embodiment of the invention encompasses a method of treating, managing, or preventing CIPN which comprises administering a therapeutically or prophylactically effective amount of an AAK1 inhibitor to the patient.

This invention further encompasses a method of treating cancer while minimizing CIPN, which comprises administering to a patient in need thereof a therapeutically effective amount of a chemotherapeutic agent and a therapeutically or prophylactically effective amount of an AAK1 inhibitor.

This invention further encompasses pharmaceutical compositions comprising a chemotherapeutic agent and an AAK1 inhibitor. Also encompassed are kits comprising drugs, which drugs comprise a chemotherapeutic agent and an AAK1 inhibitor, and instructions regarding preparation (e.g., dilution, reconstitution, dissolution) and/or administration (e.g., infusion) of the drugs.

In preferred methods, compositions, and kits of the invention, the AAK1 inhibitor is a compound of Formula I:

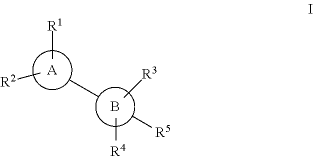

the substituents of which are defined below. In particularly preferred embodiments, the AAK1 inhibitor is (S)-1-((2′,6-bis(difluoromethyl)-[2,4′-bipyridin]-5-yl)oxy)-2,4-dimethylpentan-2-amine or a pharmaceutically acceptable salt thereof. A particular salt is ((S)-1-((2′,6-bis(difluoromethyl)-[2,4′-bipyridin]-5-yl)oxy)-2,4-dimethylpentan-2-aminium dihydrogen phosphate.

### BRIEF DESCRIPTION OF THE DRAWINGS

Certain aspects of the invention may be understood with reference to the accompanying figures, which present results obtained in a rat CIPN model (data expressed as mean±s.e.m.).

FIG. 1 shows the pre-induction and post-induction effect of a single oral administration of Compound A in a model of oxaliplatin-induced peripheral neuropathy in rats.

FIG. 2 provides Table 2.

FIG. 3 shows the effect of a single oral administration of Compound A in the CIPN model 60 minutes after treatment.

FIG. 4 provides Table 3.

FIG. 5 shows the effect of repeated administrations of Compound A at two doses in the CIPN model in rats at baseline.

FIG. 6 provides Table 4.

FIG. 7 shows the effect of repeated administrations of Compound A at two doses in the CIPN model 60 minutes after treatment.

FIG. 8 provides Table 5.

### DETAILED DESCRIPTION

This invention is based on the discovery that the inhibition of adaptor associated kinase 1 (AAK1) (e.g., by the administration of a centrally-acting AAK1 inhibitor) can reduce chemotherapy-induced pain.

This invention is directed to methods of treating, managing, and preventing CIPN, which comprise inhibiting AAK1 in a patient in need thereof. A preferred embodiment encompasses a method of treating, managing, or preventing CIPN which comprises administering a therapeutically or prophylactically effective amount of an AAK1 inhibitor to the patient.

In some embodiments of the invention, the CIPN is chronic. In some, the CIPN is acute. Chronic CIPN is pain that occurs during or shortly after administration of a chemotherapeutic agent and lasts up to seven days following the administration. Chronic pain may last for years following chemotherapy, and can occur in patients who are free of cancer.

In some embodiments, the patient is undergoing chemotherapy, which chemotherapy comprises administering to the patient a chemotherapeutic agent.

This invention also encompasses a method of treating cancer while minimizing CIPN, which comprises administering to a patient in need thereof a therapeutically effective amount of a chemotherapeutic agent and a therapeutically or prophylactically effective amount of an AAK1 inhibitor.

In some embodiments of the invention, the AAK1 inhibitor is administered to the patient before (e.g., within 1, 2, 4, 6, 12, 24, or 48 hours before) the administration of the chemotherapeutic agent. In some embodiments, the AAK1 inhibitor and chemotherapeutic agent are administered concurrently to the patient. In some embodiments, the AAK1 inhibitor is administered to the patient after (e.g., within 1, 2, 4, 6, 12, or 24 hours following) the administration of the chemotherapeutic agent. In some embodiments, the chemotherapeutic agent and the AAK1 inhibitor are administered to the patient by the same route of administration (e.g., infusion).

In some embodiments, the patient has cancer. In some embodiments, the cancer is colorectal cancer, neuroblastoma, biliary adenocarcinoma, leukemia, esophageal cancer, gastric cancer, neuroendocrine tumors, non-Hodgkin lymphoma, pancreatic cancer, ovarian cancer, or testicular cancer. In some embodiments, the cancer is colorectal cancer (e.g., metastatic colorectal cancer).

In some embodiments, the chemotherapeutic agent is a platinum-based drug (e.g., chelated platinum such as oxaliplatin and cisplatin), a taxane, a proteosome inhibitor, a vinca alkaloid, or an immunomodulatory drug. In some embodiments, the chemotherapeutic agent is 5-fluorouracil, capecitabine, cisplatin, cytarabine, docetaxel, doxorubicin, epirubicin, etoposide, fludarabine, gemcitabine, ifosfamide, irinotecan, leucovorin, oxaliplatin, paclitaxel, or rituximab. In some embodiments, the chemotherapeutic agent is chelated platinum.

A particular embodiment of this invention is directed to a method of treating, managing, or preventing chemotherapy-induced pain, which comprises administering a therapeutically or prophylactically effective amount of an AAK1 inhibitor to a patient in need thereof, wherein the AAK1inhibitor is (S)-1-((2′,6-bis(difluoromethyl)-[2,4′-bipyridin]-5-yl)oxy)-2,4-dimethylpentan-2-amine or a pharmaceutically acceptable salt thereof. In some instances, the AAK1 inhibitor is ((S)-1-((2′,6-bis(difluoromethyl)-[2,4′-bipyridin]-5-yl)oxy)-2,4-dimethylpentan-2-aminium dihydrogen phosphate.

A particular embodiment of this invention is directed to a method of treating cancer while minimizing chemotherapy-induced pain, which comprises administering to a patient in need thereof a therapeutically effective amount of a chemotherapeutic agent and a therapeutically or prophylactically effective amount of an AAK1 inhibitor, wherein the chemotherapeutic agent is chelated platinum and the AAK1 inhibitor is (S)-1-((2′,6-bis(difluoromethyl)-[2,4′-bipyridin]-5-yl)oxy)-2,4-dimethylpentan-2-amine or a pharmaceutically acceptable salt thereof. In some instances, the chelated platinum is oxaliplatin. In some instances, the AAK1 inhibitor is ((S)-1-((2′,6-bis(difluoromethyl)-[2,4′-bipyridin]-5-yl)oxy)-2,4-dimethylpentan-2-aminium dihydrogen phosphate.

In some methods of the invention, the patient no longer has cancer, but still suffers from CIPN.

Some methods further comprise administering to the patient a therapeutically or prophylactically effective amount of a selective serotonin and norepinephrine reuptake inhibitor (e.g., duloxetine).

This invention further encompasses pharmaceutical compositions (e.g., tablets, capsules, solutions suitable for intravenous infusion, solids suitable for reconstitution for administration) comprising a chemotherapeutic agent and an AAK1 inhibitor.

This invention also encompasses kits comprising drugs, which drugs comprise a chemotherapeutic agent and an AAK1 inhibitor, and instructions regarding the preparation (e.g., dilution, reconstitution, dissolution) and/or administration (e.g., infusion) of the drugs.

In some compositions and kits of the invention, the chemotherapeutic agent is chelated platinum, a taxane, a proteosome inhibitor, a vinca alkaloid, or an immunomodulatory drug. In some embodiments of the inventions, the chemotherapeutic agent is 5-fluorouracil, capecitabine, cisplatin, cytarabine, docetaxel, doxorubicin, epirubicin, etoposide, fludarabine, gemcitabine, ifosfamide, irinotecan, leucovorin, oxaliplatin, paclitaxel, or rituximab. In some embodiments, the chemotherapeutic agent is chelated platinum (e.g., oxaliplatin, cisplatin). Some compositions and kits further comprises a selective serotonin and norepinephrine reuptake inhibitor (e.g., duloxetine).

A particular composition comprises a chemotherapeutic agent and an AAK1 inhibitor, wherein the chemotherapeutic agent is chelated platinum and the AAK1 inhibitor is (S)-1-((2′,6-bis(difluoromethyl)-[2,4′-bipyridin]-5-yl)oxy)-2,4-dimethylpentan-2-amine or a pharmaceutically acceptable salt thereof. In certain embodiments, the chelated platinum is oxaliplatin. In certain embodiments, the AAK1 inhibitor is ((S)-1-((2′,6-bis(difluoromethyl)-[2,4′-bipyridin]-5-yl)oxy)-2,4-dimethylpentan-2-aminium dihydrogen phosphate.

A particular kit of the invention comprises drugs, which drugs comprise a chemotherapeutic agent and an AAK1 inhibitor, and instructions regarding the preparation (e.g., dilution, reconstitution, dissolution) and/or administration (e.g., infusion) of the drugs, wherein the chemotherapeutic agent is chelated platinum and the AAK1 inhibitor is (S)-1-((2′,6-bis(difluoromethyl)-[2,4′-bipyridin]-5-yl)oxy)-2,4-dimethylpentan-2-amine or a pharmaceutically acceptable salt thereof. In certain embodiments, the chelated platinum is oxaliplatin. In certain embodiments, the AAK1 inhibitor is ((S)-1-((2′,6-bis(difluoromethyl)-[2,4′-bipyridin]-5-yl)oxy)-2,4-dimethylpentan-2-aminium dihydrogen phosphate.

This invention further encompasses compositions comprising a chemotherapeutic agent and an AAK1 inhibitor, wherein the chemotherapeutic agent is chelated platinum and the AAK1 inhibitor is (S)-1-((2′,6-bis(difluoromethyl)-[2,4′-bipyridin]-5-yl)oxy)-2,4-dimethylpentan- 2-amine or a pharmaceutically acceptable salt thereof. In some instances, the AAK1 inhibitor is ((S)-1-((2′,6-bis(difluoromethyl)-[2,4′-bipyridin]-5-yl)oxy)-2,4-dimethylpentan-2-aminium dihydrogen phosphate.

Some compositions and kits further comprise a selective serotonin and norepinephrine reuptake inhibitor (e.g., duloxetine).

The terms “chemotherapy-induced pain” and “chemotherapy-induced neuropathic pain” are used interchangeably to refer to peripheral neuropathy that occurs due to chemotherapy treatment. Sensory manifestations include paresthesia (i.e., feelings of burning, tingling, or pins-and-needles), hyperpathia, and hypoesthesia. Hyperpathia is increased sensitivity to stimuli in which normal touch can be painful (allodynia); hypoesthesia is decreased sensitivity, feeling more like numbness. Motor symptoms can present as weakness, atrophy, and decreased reflexes.

Unless otherwise indicated, the terms “manage,” “managing” and “management” encompass preventing the recurrence of the specified disease or disorder in a patient who has already suffered from the disease or disorder, and/or lengthening the time that a patient who has suffered from the disease or disorder remains in remission. The terms encompass modulating the threshold, development and/or duration of the disease or disorder, or changing the way that a patient responds to the disease or disorder.

Unless otherwise indicated, the terms “prevent,” “preventing” and “prevention” contemplate an action that occurs before a patient begins to suffer from the specified disease or disorder, which inhibits or reduces the severity of the disease or disorder. In other words, the terms encompass prophylaxis.

Unless otherwise indicated, a “prophylactically effective amount” of a compound is an amount sufficient to prevent a disease or condition, or one or more symptoms associated with the disease or condition, or prevent its recurrence. A “prophylactically effective amount” of a compound means an amount of therapeutic agent, alone or in combination with other agents, which provides a prophylactic benefit in the prevention of the disease. The term “prophylactically effective amount” can encompass an amount that improves overall prophylaxis or enhances the prophylactic efficacy of another prophylactic agent.

Unless otherwise indicated, a “therapeutically effective amount” of a compound is an amount sufficient to provide a therapeutic benefit in the treatment or management of a disease or condition, or to delay or minimize one or more symptoms associated with the disease or condition. A “therapeutically effective amount” of a compound means an amount of therapeutic agent, alone or in combination with other therapies, that provides a therapeutic benefit in the treatment or management of the disease or condition. The term “therapeutically effective amount” can encompass an amount that improves overall therapy, reduces or avoids symptoms or causes of a disease or condition, or enhances the therapeutic efficacy of another therapeutic agent.

Unless otherwise indicated, the terms “treat,” “treating” and “treatment” contemplate an action that occurs while a patient is suffering from the specified disease or disorder, which reduces the severity of the disease or disorder, or retards or slows the progression of the disease or disorder. As used herein, the term encompasses the management of a disease or disorder.

### Inhibitors of Adaptor Associated Kinase 1

Adaptor associated kinase 1 is a member of the Ark1/Prk1 family of serine/threonine kinases. A number of AAK1 inhibitors have been disclosed that may be useful in the treatment of neuropathic pain. See, e.g., U.S. Pat. No. 9,902,722; Hartz, R. A., et al., J. Med. Chem., 2021 Aug. 12;64(15):11090-11128; Luo G., et al., J Med Chem. 2022 Mar. 24;65(6):4534-4564; and Luo G, et al., J Med Chem. 2022 Mar. 24;65(6):4457-4480. However, the mechanism by which the compounds exhibit their pharmacological effect remains unclear. In other words, while the compounds are effective in some pain models, exactly how inhibition of AAK1 relieves of neuropathic pain remains to be explored.

This invention encompasses methods of using and compositions comprising AAK1 inhibitors disclosed in U.S. Pat. No. 9,902,722. Particular compounds include those of formula I:

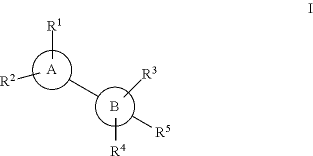

and pharmaceutically acceptable salts thereof, wherein:
A is selected from

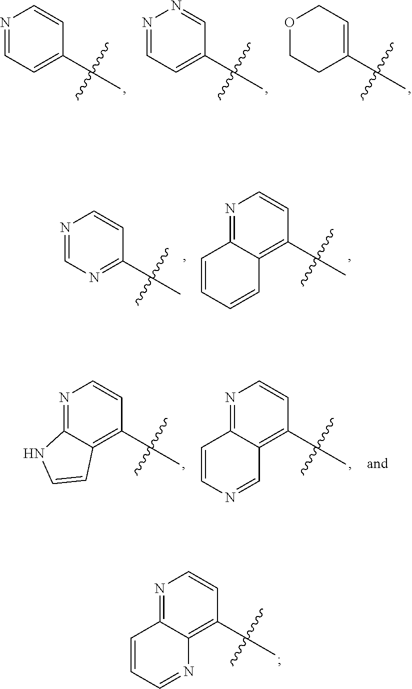

wherein [symbol] denotes the point of attachment to B;
B is selected from

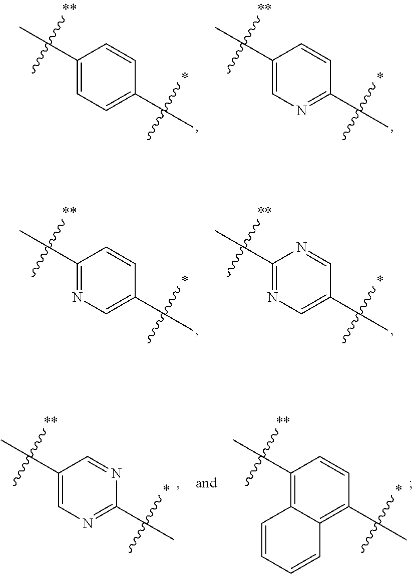 [SMILES: *c1ccc(*)cc1 ; *c1ccc(*)nc1 ; *c1ccc(*)nc1 ; *c1cnc(*)nc1 ; *c1cnc(*)nc1 ; *c1ccc(*)c2ccccc12]

wherein “*” indicates the point of attachment to R⁵ and “**” indicates the point of attachment to ring A;
R¹ is selected from hydrogen, amino, —CO₂H, difluoromethyl, ethyl, halo, hydroxymethyl, methoxy, methyl, —NHC(O)CH₃, —NHCO₂CH₃, trifluoromethoxy, and trifluoromethyl;
R² is selected from hydrogen, cyano, —CH₂OH, halo, and methyl;
R³ is selected from hydrogen, cyano, cyclopropyl, difluoromethyl, halo, hydroxymethyl, methoxy, methyl, methylsulfonyl, trifluoromethoxy, trifluoromethyl, —CH₂N(CH₃)₂, and a five-membered aromatic ring containing one, two, or three heteroatoms selected from nitrogen, oxygen, and sulfur;
R⁴ is selected from hydrogen, halo, and methyl;
R⁵ is selected from

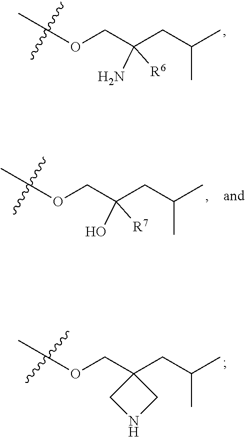

R⁶ is selected from hydrogen, ethyl, fluoromethyl, difluoromethyl, methyl, and trifluoromethyl; and
R⁷ is methyl.

In some embodiments of formula (I), A is selected from

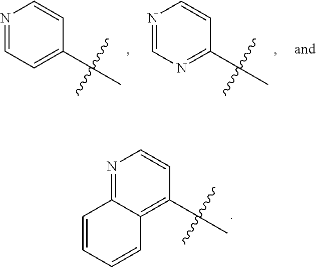 [SMILES: *c1ccncc1 ; *c1ccncn1 ; *c1ccnc2ccccc12]

In some embodiments of formula (I), B is selected from

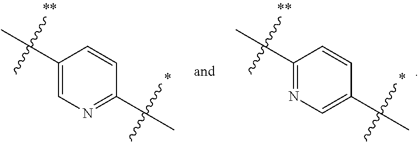 [SMILES: *c1ccc(*)nc1 ; *c1ccc(*)nc1]

In some embodiments of formula (I), B is:

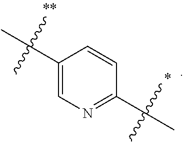 [SMILES: *c1ccc(*)nc1]

In some embodiments of formula (I), R⁵ is

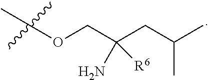

Particular AAK1 inhibitors include those of formula I):

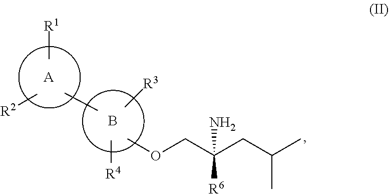

and pharmaceutically acceptable salts thereof, wherein:
A is selected from

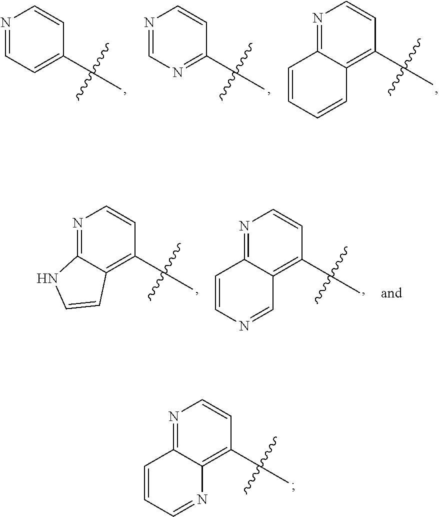

wherein [symbol] denotes the point of attachment to B;
B is selected from phenyl and pyridinyl;
R¹ is selected from hydrogen, difluoromethyl, halo, methoxy, methyl, —NHC(O)CH₃, —NHCO₂CH₃, and trifluoromethyl;
R² is selected from hydrogen, —CH₂OH, and halo;
R³ is selected from hydrogen, cyano, cyclopropyl, difluoromethyl, halo, hydroxymethyl, methoxy, methyl, trifluoromethoxy, trifluoromethyl, and a five-membered aromatic ring containing one, two, or three heteroatoms selected from nitrogen, oxygen, and sulfur;
R⁴ is selected from hydrogen, halo, and methyl; and
R⁶ is selected from hydrogen, ethyl, fluoromethyl, difluoromethyl, methyl, and trifluoromethyl.

In some embodiments of formula (II), A is selected from

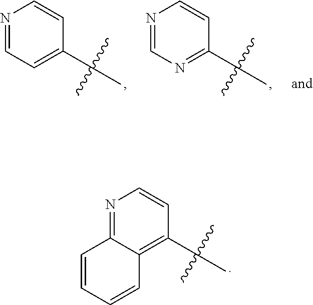 [SMILES: *c1ccncc1 ; *c1ccncn1 ; *c1ccnc2ccccc12]

In some embodiments of formula (II), B is pyridinyl.

In some embodiments of formula (II), B is:

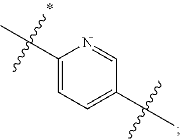

wherein [symbol] denotes the point of attachment to A and [symbol] denotes the point of attachment to the oxygen atom.

In some embodiments of formula (II), A is selected from

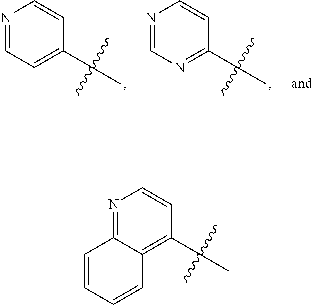 [SMILES: *c1ccncc1 ; *c1ccncn1 ; *c1ccnc2ccccc12]

B is

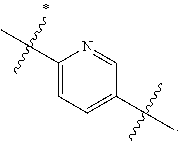 [SMILES: *c1ccc(*)nc1]

Specific AAK1 inhibitors include:
(S)-N-(4-(4-((2-amino-4-methylpentyl)oxy)-3-fluorophenyl)pyridin-2-yl)acetamide;
(S)-N-(4-(4-((2-amino-4-methylpentyl)oxy)-3-methoxyphenyl)pyridin-2-yl)acetamide;
(S)-N-(4-(4-((2-amino-4-methylpentyl)oxy)-3-cyanophenyl)pyridin-2-yl)acetamide;
(S)-2-((2-amino-4-methylpentyl)oxy)-5-(2-aminopyridin-4-yl)benzonitrile;
(S)-N-(4-(4-((2-amino-4-methylpentyl)oxy)-3-(trifluoromethyl)phenyl)pyridin-2-yl)acetamide;
(S)-N-(4-(4-((2-amino-4-methylpentyl)oxy)-3-(trifluoromethoxy)phenyl)pyridin-2-yl)acetamide;
(S)-N-(4-(4-((2-amino-4-methylpentyl)oxy)-3-methylphenyl)pyridin-2-yl)acetamide;
(S)-N-(4-(4-((2-amino-4-methylpentyl)oxy)-3-chlorophenyl)pyridin-2-yl)acetamide;
(S)-N-(4-(4-((2-amino-4-methylpentyl)oxy)-3,5-difluorophenyl)pyridin-2-yl)acetamide;
(S)-N-(4-(4-((2-amino-4-methylpentyl)oxy)-3-chloro-5-fluorophenyl)pyridin-2-yl)acetamide;
(S)-N-(4-(4-((2-amino-4-methylpentyl)oxy)-3-fluoro-5-(trifluoromethyl)phenyl)pyridin-2-yl)acetamide;
(S)-N-(4-(4-((2-amino-4-methylpentyl)oxy)-2,5-difluorophenyl)pyridin-2-yl)acetamide;
(S)-methyl(4-(4-((2-amino-4-methylpentyl)oxy)-3-fluorophenyl)pyridin-2-yl)carbamate;
(S)-methyl(4-(4-((2-amino-4-methylpentyl)oxy)-3-(isoxazol-5-yl)phenyl)pyridin-2-yl)carbamate;
(S)-2-((2-amino-4-methylpentyl)oxy)-5-(2-methylpyridin-4-yl)benzonitrile;
(S)-2-((2-amino-4-methylpentyl)oxy)-5-(2-methoxypyridin-4-yl)benzonitrile;
(S)-2-((2-amino-4-methylpentyl)oxy)-5-(2-(trifluoromethyl)pyridin-4-yl)benzonitrile;
(S)-1-(2-(isoxazol-5-yl)-4-(2-methylpyridin-4-yl)phenoxy)-4-methylpentan-2-amine;
(S)-N-(4-(4-((2-amino-2,4-dimethylpentyl)oxy)-3-(trifluoromethyl)phenyl)pyridin-2-yl)acetamide;
(S)-methyl(4-(4-((2-amino-2,4-dimethylpentyl)oxy)-3-(trifluoromethyl)phenyl)pyridin-2-yl)carbamate;
(S)-N-(4-(4-((2-amino-2,4-dimethylpentyl)oxy)-3-cyanophenyl)pyridin-2-yl)acetamide;
(S)-methyl(4-(4-((2-amino-2,4-dimethylpentyl)oxy)-3-cyanophenyl)pyridin-2-yl)carbamate;
(S)-N-(4-(4-((2-amino-2,4-dimethylpentyl)oxy)-3-(difluoromethyl)phenyl)pyridin-2-yl)acetamide;
(S)-methyl(4-(4-((2-amino-2,4-dimethylpentyl)oxy)-3-(difluoromethyl)phenyl)pyridin-2-yl)carbamate;
(S)-N-(4-(4-((2-amino-2,4-dimethylpentyl)oxy)-3-(trifluoromethoxy)phenyl)pyridin-2-yl)acetamide;
(S)-methyl(4-(4-((2-amino-2,4-dimethylpentyl)oxy)-3-(trifluoromethoxy)phenyl)pyridin-2-yl)carbamate;
(S)-N-(4-(4-((2-amino-2,4-dimethylpentyl)oxy)-3-fluorophenyl)pyridin-2-yl)acetamide;
(S)-methyl(4-(4-((2-amino-2,4-dimethylpentyl)oxy)-3-fluorophenyl)pyridin-2-yl)carbamate;
methyl(4-(4-((2-amino-2,4-dimethylpentyl)oxy)-3-chlorophenyl)pyridin-2-yl)carbamate;
methyl(4-(4-((2-amino-2,4-dimethylpentyl)oxy)-3-methylphenyl)pyridin-2-yl)carbamate;
methyl(4-(4-((2-amino-2,4-dimethylpentyl)oxy)-2,3-dimethylphenyl)pyridin-2-yl)carbamate;
(S)-methyl(4-(4-((2-amino-2,4-dimethylpentyl)oxy)-3-(hydroxymethyl)phenyl)pyridin-2-yl)carbamate;
(S)-methyl(4-(4-((2-amino-2,4-dimethylpentyl)oxy)-3-cyclopropylphenyl)pyridin-2-yl)carbamate;
(S)-N-(4-(4-((2-amino-2,4-dimethylpentyl)oxy)-3-(trifluoromethyl)phenyl)-5-(hydroxymethyl)pyridin-2-yl)acetamide;
(S)-1-(4-(2-(difluoromethyl)pyridin-4-yl)-2-(trifluoromethyl)phenoxy)-2,4-dimethylpentan-2-amine;
(S)-2-((2-amino-2,4-dimethylpentyl)oxy)-5-(2-(difluoromethyl)pyridin-4-yl)benzonitrile;
(S)-1-(2-(difluoromethyl)-4-(2-(difluoromethyl)pyridin-4-yl)phenoxy)-2,4-dimethylpentan-2-amine;
(S)-1-(4-(2-(difluoromethyl)pyridin-4-yl)-2-(trifluoromethoxy)phenoxy)-2,4-dimethylpentan-2-amine;
(S)-1-(4-(3-chloro-2-fluoropyridin-4-yl)-2-(trifluoromethyl)phenoxy)-2,4-dimethylpentan-2-amine;
(S)-1-(4-(5-chloro-2-fluoropyridin-4-yl)-2-(trifluoromethyl)phenoxy)-2,4-dimethylpentan-2-amine;
(S)-1-(4-(2-fluoro-3-methylpyridin-4-yl)-2-(trifluoromethyl)phenoxy)-2,4-dimethylpentan-2-amine;
(S)-1-(4-(2,3-difluoropyridin-4-yl)-2-(trifluoromethyl)phenoxy)-2,4-dimethylpentan-2-amine;
(S)-2,4-dimethyl-1-(4-(pyridin-4-yl)-2-(trifluoromethyl)phenoxy)pentan-2-amine;
(S)-1-(4-(2-fluoropyridin-4-yl)-2-(trifluoromethyl)phenoxy)-2,4-dimethylpentan-2-amine;
(S)-2,4-dimethyl-1-(4-(2-methylpyridin-4-yl)-2-(trifluoromethyl)phenoxy)pentan-2-amine;
(S)-1-(4-(3-methoxypyridin-4-yl)-2-(trifluoromethyl)phenoxy)-2,4-dimethylpentan-2-amine;
(S)-1-(4-(3-fluoropyridin-4-yl)-2-(trifluoromethyl)phenoxy)-2,4-dimethylpentan-2-amine;
(S)-2-((2-amino-2,4-dimethylpentyl)oxy)-5-(2-methylpyridin-4-yl)benzonitrile;
(S)-1-(2-cyclopropyl-4-(2-methylpyridin-4-yl)phenoxy)-2,4-dimethylpentan-2-amine;
(S)-1-(2-(difluoromethyl)-4-(2-methylpyridin-4-yl)phenoxy)-2,4-dimethylpentan-2-amine;
methyl(6-((2-amino-2,4-dimethylpentyl)oxy)-[3,4′-bipyridin]-2′-yl)carbamate;
(S)-methyl(6-((2-amino-2,4-dimethylpentyl)oxy)-5-methyl-[3,4′-bipyridin]-2′-yl)carbamate;
(S)-methyl(6-((2-amino-2,4-dimethylpentyl)oxy)-5-cyano-[3,4′-bipyridin]-2′-yl)carbamate;
(S)-methyl(6-((2-amino-2,4-dimethylpentyl)oxy)-4-methyl-[3,4′-bipyridin]-2′-yl)carbamate;
(S)-methyl(6-((2-amino-2,4-dimethylpentyl)oxy)-5-chloro-[3,4′-bipyridin]-2′-yl)carbamate;
(S)-methyl(6-((2-amino-2,4-dimethylpentyl)oxy)-5-methoxy-[3,4′-bipyridin]-2′-yl)carbamate;
(S)-1-((2′-chloro-5-methyl-[3,4′-bipyridin]-6-yl)oxy)-2,4-dimethylpentan-2-amine;
(S)-1-((2′-(difluoromethyl)-5-methyl-[3,4′-bipyridin]-6-yl)oxy)-2,4-dimethylpentan-2-amine;
(S)-6-((2-amino-2,4-dimethylpentyl)oxy)-2′-(difluoromethyl)-[3,4′-bipyridine]-5-carbonitrile;
(S)-1-((5-chloro-2′-methyl-[3,4′-bipyridin]-6-yl)oxy)-2,4-dimethylpentan-2-amine;
(S)-1-((2′,5-dimethyl-[3,4′-bipyridin]-6-yl)oxy)-2,4-dimethylpentan-2-amine;
(S)-1-((5-methoxy-2′-methyl-[3,4′-bipyridin]-6-yl)oxy)-2,4-dimethylpentan-2-amine;
methyl(5-((2-amino-2,4-dimethylpentyl)oxy)-[2,4′-bipyridin]-2′-yl)carbamate;
(S)-methyl(5-((2-amino-2,4-dimethylpentyl)oxy)-6-methyl-[2,4′-bipyridin]-2′-yl)carbamate;
(S)-1-((2′,6-dimethyl-[2,4′-bipyridin]-5-yl)oxy)-2,4-dimethylpentan-2-amine;
(S)-methyl(5-((2-amino-2,4-dimethylpentyl)oxy)-6-chloro-[2,4′-bipyridin]-2′-yl)carbamate;
(S)-1-((6-chloro-2′-methyl-[2,4′-bipyridin]-5-yl)oxy)-2,4-dimethylpentan-2-amine;
(S)-methyl(5-((2-amino-2,4-dimethylpentyl)oxy)-4-methyl-[2,4′-bipyridin]-2′-yl)carbamate;
(S)-2,4-dimethyl-1-(4-(quinolin-4-yl)-2-(trifluoromethyl)phenoxy)pentan-2-amine;
(S)-2,4-dimethyl-1-(2-(trifluoromethyl)-4-(7-(trifluoromethyl)quinolin-4-yl)phenoxy)pentan-2-amine;
(S)-1-(4-(7-fluoroquinolin-4-yl)-2-(trifluoromethyl)phenoxy)-2,4-dimethylpentan-2-amine;
(S)-1-(4-(5,7-difluoroquinolin-4-yl)-2-(trifluoromethyl)phenoxy)-2,4-dimethylpentan-2-amine;
(S)-1-(4-(6-fluoroquinolin-4-yl)-2-(trifluoromethyl)phenoxy)-2,4-dimethylpentan-2-amine;
(S)-1-(2-cyclopropyl-4-(quinolin-4-yl)phenoxy)-2,4-dimethylpentan-2-amine;
1-(2-chloro-6-fluoro-4-(quinolin-4-yl)phenoxy)-2,4-dimethylpentan-2-amine;
(S)-1-((5-(7-fluoroquinolin-4-yl)-3-methylpyridin-2-yl)oxy)-2,4-dimethylpentan-2-amine;
(S)-2-((2-amino-2,4-dimethylpentyl)oxy)-5-(5,7-difluoroquinolin-4-yl)nicotinonitrile;
(S)-1-((3-chloro-5-(quinolin-4-yl)pyridin-2-yl)oxy)-2,4-dimethylpentan-2-amine;
(S)-1-((3-methoxy-5-(quinolin-4-yl)pyridin-2-yl)oxy)-2,4-dimethylpentan-2-amine;
(S)-2-((2-amino-2,4-dimethylpentyl)oxy)-5-(1,6-naphthyridin-4-yl)nicotinonitrile;
(S)-2,4-dimethyl-1-((2-methyl-6-(quinolin-4-yl)pyridin-3-yl)oxy)pentan-2-amine;
(S)-2,4-dimethyl-1-((4-methyl-6-(quinolin-4-yl)pyridin-3-yl)oxy)pentan-2-amine;
(S)-1-((2-chloro-6-(quinolin-4-yl)pyridin-3-yl)oxy)-2,4-dimethylpentan-2-amine;
(S)-1-(4-(1H-pyrrolo[2,3-b]pyridin-4-yl)-2-(trifluoromethyl)phenoxy)2,4-dimethylpentan-2-amine;
(S)-1-(4-(1,6-naphthyridin-4-yl)-2-(trifluoromethyl)phenoxy)-2,4-dimethylpentan-2-amine;
(S)-2-((2-amino-2,4-dimethylpentyl)oxy)-5-(1,6-naphthyridin-4-yl)benzonitrile;
(S)-2-((2-amino-2,4-dimethylpentyl)oxy)-5-(1,5-naphthyridin-4-yl)benzonitrile;
(S)-1-(4-(7-chloroquinolin-4-yl)-2-(trifluoromethyl)phenoxy)-2,4-dimethylpentan-2-amine;
(S)-4-(4-((2-amino-2,4-dimethylpentyl)oxy)-3-(trifluoromethyl)phenyl)quinoline-7-carbonitrile;
(S)-2,4-dimethyl-1-(2-methyl-4-(2-methylpyridin-4-yl)phenoxy)pentan-2-amine;
(S)-1-(2-fluoro-4-(2-methylpyridin-4-yl)phenoxy)-2,4-dimethylpentan-2-amine;
(S)-1-(4-(2-fluoropyridin-4-yl)-2-methylphenoxy)-2,4-dimethylpentan-2-amine;
(S)-1-(2-fluoro-4-(2-fluoropyridin-4-yl)phenoxy)-2,4-dimethylpentan-2-amine;
(S)-2-((2-amino-2,4-dimethylpentyl)oxy)-5-(2-fluoropyridin-4-yl)benzonitrile;
(S)-1-((2′-fluoro-5-methyl-[3,4′-bipyridin]-6-yl)oxy)-2,4-dimethylpentan-2-amine;
(S)-4-(6-((2-amino-2,4-dimethylpentyl)oxy)-5-methylpyridin-3-yl)quinoline-7-carbonitrile;
(S)-1-((5-fluoro-2′-methyl-[3,4′-bipyridin]-6-yl)oxy)-2,4-dimethylpentan-2-amine;
(S)-1-((3-fluoro-5-(quinolin-4-yl)pyridin-2-yl)oxy)-2,4-dimethylpentan-2-amine;
(S)-4-(6-((2-amino-2,4-dimethylpentyl)oxy)-5-fluoropyridin-3-yl)quinoline-7-carbonitrile;
(S)-methyl(4-(4-((2-amino-2,4-dimethylpentyl)oxy)-3-cyanophenyl)-3-fluoropyridin-2-yl)carbamate;
(S)-6-((2-amino-2,4-dimethylpentyl)oxy)-2′-methyl-[3,4′-bipyridine]-5-carbonitrile;
(S)-2-((2-amino-2,4-dimethylpentyl)oxy)-5-(7-methylquinolin-4-yl)benzonitrile;
(S)-2-((2-amino-2,4-dimethylpentyl)oxy)-5-(3-fluoro-2-methylpyridin-4-yl)benzonitrile;
(S)-2-((2-amino-2,4-dimethylpentyl)oxy)-5-(quinolin-4-yl)benzonitrile;
(S)-2-((2-amino-2,4-dimethylpentyl)oxy)-5-(5-fluoro-2-methylpyridin-4-yl)benzonitrile;
(S)-methyl(4-(4-((2-amino-2,4-dimethylpentyl)oxy)-3-cyanophenyl)-5-fluoropyridin-2-yl)carbamate;
(S)-1-((6-fluoro-2′,4-dimethyl-[2,4′-bipyridin]-5-yl)oxy)-2,4-dimethylpentan-2-amine;
methyl(5-((3-isobutylazetidin-3-yl)methoxy)-6-methyl-[2,4′-bipyridin]-2′-yl)carbamate;
(S)-2-((2-amino-4-methylpentyl)oxy)-5-(6-methylpyridazin-4-yl)benzonitrile;
(S)-1-(2-(isoxazol-5-yl)-4-(quinolin-4-yl)phenoxy)-4-methylpentan-2-amine;
(S)-4-(4-((2-amino-2,4-dimethylpentyl)oxy)-3-(trifluoromethyl)phenyl)-2-methylnicotinic acid;
(S)-methyl(4-(4-((2-amino-2,4-dimethylpentyl)oxy)-3-((dimethylamino)methyl)phenyl)pyridin-2-yl)carbamate;
(S)-methyl(4-(4-((2-amino-2,4-dimethylpentyl)oxy)-3-(methylsulfonyl)phenyl)pyridin-2-yl)carbamate;
(S)-2,4-dimethyl-1-(4-(2-methylpyridin-4-yl)-2-(methylsulfonyl)phenoxy)pentan-2-amine;
(S)-2,4-dimethyl-1-(2-(methylsulfonyl)-4-(quinolin-4-yl)phenoxy)pentan-2-amine;
(S)-1-(2-(difluoromethyl)-4-(6-fluoroquinolin-4-yl)phenoxy)-2,4-dimethylpentan-2-amine;
(S)-methyl(5-((2-amino-2,4-dimethylpentyl)oxy)-6-(difluoromethyl)-[2,4′-bipyridin]-2′-yl)carbamate;
(S)-1-((2-(difluoromethyl)-6-(quinolin-4-yl)pyridin-3-yl)oxy)-2,4-dimethylpentan-2-amine;
(S)-1-((6-(difluoromethyl)-2′-methyl-[2,4′-bipyridin]-5-yl)oxy)-2,4-dimethylpentan-2-amine;
(S)-methyl(5-((2-amino-2,4-dimethylpentyl)oxy)-4-(difluoromethyl)-[2,4′-bipyridin]-2′-yl)carbamate;
(S)-1-((4-(difluoromethyl)-6-(quinolin-4-yl)pyridin-3-yl)oxy)-2,4-dimethylpentan-2-amine;
(S)-1-((4-(difluoromethyl)-2′-methyl-[2,4′-bipyridin]-5-yl)oxy)-2,4-dimethylpentan-2-amine;
(S)-1-((2′,6-bis(difluoromethyl)-[2,4′-bipyridin]-5-yl)oxy)-2,4-dimethylpentan-2-amine;
(S)-1-((2′,4-bis(difluoromethyl)-[2,4′-bipyridin]-5-yl)oxy)-2,4-dimethylpentan-2-amine;
(S)-1-((2′-(difluoromethyl)-4-methyl-[2,4′-bipyridin]-5-yl)oxy)-2,4-dimethylpentan-2-amine;
(S)-1-(2-cyclopropyl-4-(2-(difluoromethyl)pyridin-4-yl)phenoxy)-2,4-dimethylpentan-2-amine;
(S)-1-((2-(difluoromethyl)-6-(6-fluoroquinolin-4-yl)pyridin-3-yl)oxy)-2,4-dimethylpentan-2-amine;
(S)-1-((2-(difluoromethyl)-6-(7-fluoroquinolin-4-yl)pyridin-3-yl)oxy)-2,4-dimethylpentan-2-amine;
(S)-1-((2-(difluoromethyl)-6-(5,7-difluoroquinolin-4-yl)pyridin-3-yl)oxy)-2,4-dimethylpentan-2-amine;
(S)-1-((4-(difluoromethyl)-6-(5,7-difluoroquinolin-4-yl)pyridin-3-yl)oxy)-2,4-dimethylpentan-2-amine;
(S)-1-((4-(difluoromethyl)-6-(7-fluoroquinolin-4-yl)pyridin-3-yl)oxy)-2,4-dimethylpentan-2-amine;
(S)-1-((4-(difluoromethyl)-6-(6-fluoroquinolin-4-yl)pyridin-3-yl)oxy)-2,4-dimethylpentan-2-amine;
((S)-1-((4-(difluoromethyl)-6-(6-(trifluoromethyl)quinolin-4-yl)pyridin-3-yl)oxy)-2,4-dimethylpentan-2-amine;
(S)-1-((4-(difluoromethyl)-6-(6-(trifluoromethoxy)quinolin-4-yl)pyridin-3-yl)oxy)-2,4-dimethylpentan-2-amine;
(S)-1-((2-chloro-6-(5,7-difluoroquinolin-4-yl)pyridin-3-yl)oxy)-2,4-dimethylpentan-2-amine;
(S)-1-((2-chloro-6-(7-fluoroquinolin-4-yl)pyridin-3-yl)oxy)-2,4-dimethylpentan-2-amine;
(S)-1-((6-(7-fluoroquinolin-4-yl)-2-methylpyridin-3-yl)oxy)-2,4-dimethylpentan-2-amine;
(S)-1-((6-(6-fluoroquinolin-4-yl)-2-methylpyridin-3-yl)oxy)-2,4-dimethylpentan-2-amine;
(S)-1-((2-(difluoromethyl)-6-(2-methylpyrimidin-4-yl)pyridin-3-yl)oxy)-2,4-dimethylpentan-2-amine;
(S)-1-((2-(difluoromethyl)-6-(6-methylpyrimidin-4-yl)pyridin-3-yl)oxy)-2,4-dimethylpentan-2-amine;
(S)-1-((4-(difluoromethyl)-2′-ethyl-[2,4′-bipyridin]-5-yl)oxy)-2,4-dimethylpentan-2-amine;
(S)-1-((2′-chloro-4-(difluoromethyl)-3′-fluoro-[2,4′-bipyridin]-5-yl)oxy)-2,4-dimethylpentan-2-amine;
(S)-1-((2′-chloro-4-(difluoromethyl)-5′-fluoro-[2,4′-bipyridin]-5-yl)oxy)-2,4-dimethylpentan-2-amine;
(S)-2,4-dimethyl-1-((2′-methyl-4-(trifluoromethyl)-[2,4′-bipyridin]-5-yl)oxy)pentan-2-amine;
(S)-2,4-dimethyl-1-((6-(quinolin-4-yl)-4-(trifluoromethyl)pyridin-3-yl)oxy)pentan-2-amine;
(S)-methyl(5-((2-amino-2,4-dimethylpentyl)oxy)-4-(trifluoromethyl)-[2,4′-bipyridin]-2′-yl)carbamate;
(S)-1-((2′-chloro-4-(difluoromethyl)-[2,4′-bipyridin]-5-yl)oxy)-2,4-dimethylpentan-2-amine;
(S)-1-((4-(difluoromethyl)-5′-fluoro-2′-methyl-[2,4′-bipyridin]-5-yl)oxy)-2,4-dimethylpentan-2-amine;
(S)-1-((4-(difluoromethyl)-3′-fluoro-2′-methyl-[2,4′-bipyridin]-5-yl)oxy)-2,4-dimethylpentan-2-amine;
(S)-1-((4-(difluoromethyl)-6-(2-methylpyrimidin-4-yl)pyridin-3-yl)oxy)-2,4-dimethylpentan-2-amine;
(S)-1-(2-(difluoromethyl)-4-(2-methylpyrimidin-4-yl)phenoxy)-2,4-dimethylpentan-2-amine;
(S)-5-((2-amino-2,4-dimethylpentyl)oxy)-3′-fluoro-6-methyl-[2,4′-bipyridin]-2′-amine;
(S)-1-((2′-chloro-3′-fluoro-6-methyl-[2,4′-bipyridin]-5-yl)oxy)-2,4-dimethylpentan-2-amine;
(S)-methyl(5-((2-amino-2,4-dimethylpentyl)oxy)-5′-fluoro-6-methyl-[2,4′-bipyridin]-2′-yl)carbamate;
(S)-1-((2′-chloro-5′-fluoro-6-methyl-[2,4′-bipyridin]-5-yl)oxy)-2,4-dimethylpentan-2-amine;
(S)-methyl(5-((2-amino-2,4-dimethylpentyl)oxy)-3′-fluoro-6-methyl-[2,4′-bipyridin]-2′-yl)carbamate;
(S)-1-((2′-chloro-6-(difluoromethyl)-[2,4′-bipyridin]-5-yl)oxy)-2,4-dimethylpentan-2-amine;
(S)-1-((2′-chloro-6-(difluoromethyl)-3′-fluoro-[2,4′-bipyridin]-5-yl)oxy)-2,4-dimethylpentan-2-amine;
(S)-1-((6-(difluoromethyl)-3′-fluoro-2′-methyl-[2,4′-bipyridin]-5-yl)oxy)-2,4-dimethylpentan-2-amine;
((S)-1-((6-chloro-2′-(difluoromethyl)-[2,4′-bipyridin]-5-yl)oxy)-2,4-dimethylpentan-2-amine;
(S)-1-((4-(difluoromethyl)-6-(6-methylpyridazin-4-yl)pyridin-3-yl)oxy)-2,4-dimethylpentan-2-amine;
(S)-1-((2-(difluoromethyl)-6-(6-methylpyridazin-4-yl)pyridin-3-yl)oxy)-2,4-dimethylpentan-2-amine;
(R)-2,4-dimethyl-1-((2′-methyl-4-(trifluoromethyl)-[2,4′-bipyridin]-5-yl)oxy)pentan-2-amine;
(R)-2,4-dimethyl-1-((6-(quinolin-4-yl)-4-(trifluoromethyl)pyridin-3-yl)oxy)pentan-2-amine;
(R)-methyl(5-((2-amino-2,4-dimethylpentyl)oxy)-4-(trifluoromethyl)-[2,4′-bipyridin]-2′-yl)carbamate;
(R)-1-((2′-chloro-4-(difluoromethyl)-3′-fluoro-[2,4′-bipyridin]-5-yl)oxy)-2,4-dimethylpentan-2-amine;
(R)-1-((2′-chloro-4-(difluoromethyl)-5′-fluoro-[2,4′-bipyridin]-5-yl)oxy)-2,4-dimethylpentan-2-amine;
(R)-1-((2′-chloro-4-(difluoromethyl)-[2,4′-bipyridin]-5-yl)oxy)-2,4-dimethylpentan-2-amine;
(R)-1-((4-(difluoromethyl)-2′-ethyl-[2,4′-bipyridin]-5-yl)oxy)-2,4-dimethylpentan-2-amine;
(R)-1-((4-(difluoromethyl)-5′-fluoro-2′-methyl-[2,4′-bipyridin]-5-yl)oxy)-2,4-dimethylpentan-2-amine;
(R)-1-((4-(difluoromethyl)-3′-fluoro-2′-methyl-[2,4′-bipyridin]-5-yl)oxy)-2,4-dimethylpentan-2-amine;
(R)-methyl(5-((2-amino-2,4-dimethylpentyl)oxy)-6-methyl-[2,4′-bipyridin]-2′-yl)carbamate;
(R)-1-((2′,6-bis(difluoromethyl)-[2,4′-bipyridin]-5-yl)oxy)-2,4-dimethylpentan-2-amine;
(R)-1-((2′,4-bis(difluoromethyl)-[2,4′-bipyridin]-5-yl)oxy)-2,4-dimethylpentan-2-amine;
(S)-methyl(4-(difluoromethyl)-5-((2-hydroxy-2,4-dimethylpentyl)oxy)-[2,4′-bipyridin]-2′-yl)carbamate;
(S)-1-((4-(difluoromethyl)-2′-methyl-[2,4′-bipyridin]-5-yl)oxy)-2,4-dimethylpentan-2-ol;
(S)-methyl(5-((2-amino-2-(fluoromethyl)-4-methylpentyl)oxy)-6-(difluoromethyl)-[2,4′-bipyridin]-2′-yl)carbamate;
(R)-methyl(5-((2-amino-2-(fluoromethyl)-4-methylpentyl)oxy)-6-(difluoromethyl)-[2,4′-bipyridin]-2′-yl)carbamate;
(S)-methyl (5- ((2-amino-2-(fluoromethyl)-4-methylpentyl)oxy)-4-(difluoromethyl)-[2,4′-bipyridin]-2′-yl)carbamate;
(R)-methyl (5- ((2-amino-2-(fluoromethyl)-4-methylpentyl)oxy)-4-(difluoromethyl)-[2,4′-bipyridin]-2′-yl)carbamate;
(S)-1-((2′,6-bis(difluoromethyl)-[2,4′-bipyridin]-5-yl)oxy)-2-(fluoromethyl)-4-methylpentan-2-amine;
(S)-1-((2′,4-bis(difluoromethyl)-[2,4′-bipyridin]-5-yl)oxy)-2-(fluoromethyl)-4-methylpentan-2-amine;
(R)-1-((2′,6-bis(difluoromethyl)-[2,4′-bipyridin]-5-yl)oxy)-2-(fluoromethyl)-4-methylpentan-2-amine;
(R)-1-((2′,4-bis(difluoromethyl)-[2,4′-bipyridin]-5-yl)oxy)-2-(fluoromethyl)-4-methylpentan-2-amine;
(S)-1-((4-(difluoromethyl)-2′-methyl-[2,4′-bipyridin]-5-yl)oxy)-2-(fluoromethyl)-4-methylpentan-2-amine;
(R)-1-((4-(difluoromethyl)-2′-methyl-[2,4′-bipyridin]-5-yl)oxy)-2-(fluoromethyl)-4-methylpentan-2-amine;
(S)-N-(4-(4-((2-amino-4-methylpentyl)oxy)-3-fluorophenyl)pyridin-2-yl)acetamide;
(S)-1-((3-chloro-5-(7-fluoroquinolin-4-yl)pyridin-2-yl)oxy)-2,4-dimethylpentan-2-amine;
(S)-1-((3-chloro-5-(6-fluoroquinolin-4-yl)pyridin-2-yl)oxy)-2,4-dimethylpentan-2-amine;
(S)-1-((3-chloro-5-(2-methylpyrimidin-4-yl)pyridin-2-yl)oxy)-2,4-dimethylpentan-2-amine;
(S)-1-((2′-chloro-5-(difluoromethyl)-[3,4′-bipyridin]-6-yl)oxy)-2,4-dimethylpentan-2-amine;
(S)-1-((5-(difluoromethyl)-2′-methyl-[3,4′-bipyridin]-6-yl)oxy)-2,4-dimethylpentan-2-amine;
(S)-1-((3-(difluoromethyl)-5-(7-fluoroquinolin-4-yl)pyridin-2-yl)oxy)-2,4-dimethylpentan-2-amine;
(S)-methyl(6-((2-amino-2,4-dimethylpentyl)oxy)-5-(difluoromethyl)-[3,4′-bipyridin]-2′-yl)carbamate;
(S)-1-((5-(7-chloroquinolin-4-yl)-3-(difluoromethyl)pyridin-2-yl)oxy)-2,4-dimethylpentan-2-amine;
(S)-1-((3-(difluoromethyl)-5-(2-methylpyrimidin-4-yl)pyridin-2-yl)oxy)-2,4-dimethylpentan-2-amine;
(S)-1-((2′,5-bis(difluoromethyl)-[3,4′-bipyridin]-6-yl)oxy)-2,4-dimethylpentan-2-amine;
(S)-1-((3-(difluoromethyl)-5-(6-fluoroquinolin-4-yl)pyridin-2-yl)oxy)-2,4-dimethylpentan-2-amine;
(S)-1-((3-(difluoromethyl)-5-(5,7-difluoroquinolin-4-yl)pyridin-2-yl)oxy)-2,4-dimethylpentan-2-amine;
(S)-1-((3-(difluoromethyl)-5-(7-(trifluoromethyl)quinolin-4-yl)pyridin-2-yl)oxy)-2,4-dimethylpentan-2-amine;
(S)-1-((5-(difluoromethyl)-2′,3′-dimethyl-[3,4′-bipyridin]-6-yl)oxy)-2,4-dimethylpentan-2-amine;
(S)-1-((3-(difluoromethyl)-5-(quinolin-4-yl)pyridin-2-yl)oxy)-2,4-dimethylpentan-2-amine;
(S)-1-((3-(difluoromethyl)-5-(7-methylquinolin-4-yl)pyridin-2-yl)oxy)-2,4-dimethylpentan-2-amine;
(S)-methyl(5-((2-amino-2,4-dimethylpentyl)oxy)-4-methoxy-[2,4′-bipyridin]-2′-yl)carbamate;
(S)-1-((2′-(difluoromethyl)-4-methoxy-[2,4′-bipyridin]-5-yl)oxy)-2,4-dimethylpentan-2-amine;
(S)-methyl(4-(4-((2-amino-2,4-dimethylpentyl)oxy)naphthalen-1-yl)pyridin-2-yl)carbamate;
(S)-2,4-dimethyl-1-((4-(quinolin-4-yl)naphthalen-1-yl)oxy)pentan-2-amine;
(S)-methyl(4-(5-((2-amino-2,4-dimethylpentyl)oxy)pyrimidin-2-yl)pyridin-2-yl)carbamate;
(S)-methyl(4-(2-((2-amino-2,4-dimethylpentyl)oxy)pyrimidin-5-yl)pyridin-2-yl)carbamate;
(S)-2,4-dimethyl-1-((2′,4,6-trimethyl-[2,4′-bipyridin]-5-yl)oxy)pentan-2-amine;
(S)-methyl(5-((2-amino-2,4-dimethylpentyl)oxy)-4,6-dimethyl-[2,4′-bipyridin]-2′-yl)carbamate;
(S)-2,4-dimethyl-1-(4-(quinazolin-4-yl)-2-(trifluoromethyl)phenoxy)pentan-2-amine;
(S)-1-(4-(3,6-dihydro-2H-pyran-4-yl)-2-(trifluoromethyl)phenoxy)-2,4-dimethylpentan-2-amine;
(S)-2,4-dimethyl-1-(4-(2-methylquinolin-4-yl)-2-(trifluoromethyl)phenoxy)pentan-2-amine;
(S)-1-(4-(6-chloroquinolin-4-yl)-2-(trifluoromethyl)phenoxy)-2,4-dimethylpentan-2-amine;
(S)-1-((5-(5,7-difluoroquinolin-4-yl)-3-(trifluoromethyl)pyridin-2-yl)oxy)-2,4-dimethylpentan-2-amine;
(S)-2,4-dimethyl-1-((5-(quinolin-4-yl)-3-(trifluoromethyl)pyridin-2-yl)oxy)pentan-2-amine;
(S)-methyl(6-((2-amino-2,4-dimethylpentyl)oxy)-5-(trifluoromethyl)-[3,4′-bipyridin]-2′-yl)carbamate;
(S)-2,4-dimethyl-1-((2′-methyl-5-(trifluoromethyl)-[3,4′-bipyridin]-6-yl)oxy)pentan-2-amine;
(S)-1-((5-(6-chloroquinolin-4-yl)-3-methylpyridin-2-yl)oxy)-2,4-dimethylpentan-2-amine;
(S)-1-((5-(6-fluoroquinolin-4-yl)-3-methylpyridin-2-yl)oxy)-2,4-dimethylpentan-2-amine;
(S)-1-((5-(5,7-difluoroquinolin-4-yl)-3-methylpyridin-2-yl)oxy)-2,4-dimethylpentan-2-amine;
(S)-1-((5-(7-chloroquinolin-4-yl)-3-methylpyridin-2-yl)oxy)-2,4-dimethylpentan-2-amine;
(S)-1-((6-(5,7-difluoroquinolin-4-yl)-4-methylpyridin-3-yl)oxy)-2,4-dimethylpentan-2-amine;
(S)-1-((6-(7-fluoroquinolin-4-yl)-4-methylpyridin-3-yl)oxy)-2,4-dimethylpentan-2-amine;
(S)-1-((2′,4-dimethyl-[2,4′-bipyridin]-5-yl)oxy)-2,4-dimethylpentan-2-amine;
(S)-1-((6-(6-chloroquinolin-4-yl)-4-methylpyridin-3-yl)oxy)-2,4-dimethylpentan-2-amine;
(S)-1-((6-(6-fluoroquinolin-4-yl)-4-methylpyridin-3-yl)oxy)-2,4-dimethylpentan-2-amine;
(S)-1-((6-(7-chloroquinolin-4-yl)-4-methylpyridin-3-yl)oxy)-2,4-dimethylpentan-2-amine;
(S)-1-((6-(7-fluoroquinolin-4-yl)-4-methylpyridin-3-yl)oxy)-2,4-dimethylpentan-2-amine;
(S)-1-((4-chloro-6-(5,7-difluoroquinolin-4-yl)pyridin-3-yl)oxy)-2,4-dimethylpentan-2-amine;
(S)-1-((4-chloro-[2,4′-bipyridin]-5-yl)oxy)-2,4-dimethylpentan-2-amine;
(S)-1-((4-chloro-6-(6-fluoroquinolin-4-yl)pyridin-3-yl)oxy)-2,4-dimethylpentan-2-amine;
(S)-1-((4-chloro-6-(quinolin-4-yl) pyridin-3-yl)oxy)-2,4-dimethylpentan-2-amine;
(S)-methyl(5-((2-amino-2,4-dimethylpentyl)oxy)-4-chloro-[2,4′-bipyridin]-2′-yl)carbamate;
(S)-1-((4-chloro-2′-methyl-[2,4′-bipyridin]-5-yl)oxy)-2,4-dimethylpentan-2-amine;
(S)-1-((4-chloro-6-(6-chloroquinolin-4-yl)pyridin-3-yl)oxy)-2,4-dimethylpentan-2-amine;
(S)-1-((2′,4-dichloro-[2,4′-bipyridin]-5-yl)oxy)-2,4-dimethylpentan-2-amine;
(S)-1-((4-chloro-6-(7-fluoroquinolin-4-yl)pyridin-3-yl)oxy)-2,4-dimethylpentan-2-amine;
(S)-1-((4-chloro-6-(7-(trifluoromethyl)quinolin-4-yl)pyridin-3-yl)oxy)-2,4-dimethylpentan-2-amine;
(S)-1-((4-chloro-2′,3′-dimethyl-[2,4′-bipyridin]-5-yl)oxy)-2,4-dimethylpentan-2-amine;
(S)-1-((5-(difluoromethyl)-2′-methyl-[3,4′-bipyridin]-6-yl)oxy)-2,4-dimethylpentan-2-amine;
(R)-methyl(6-((2-amino-2,4-dimethylpentyl)oxy)-5-(difluoromethyl)-[3,4′-bipyridin]-2′-yl)carbamate;
(R)-1-((3-(difluoromethyl)-5-(quinolin-4-yl)pyridin-2-yl)oxy)-2,4-dimethylpentan-2-amine;
(R)-1-((3-(difluoromethyl)-5-(2-methylpyrimidin-4-yl)pyridin-2-yl)oxy)-2,4-dimethylpentan-2-amine;
(R)-1-((3-(difluoromethyl)-5-(5,7-difluoroquinolin-4-yl)pyridin-2-yl)oxy)-2,4-dimethylpentan-2-amine;
(R)-1-((5-(7-chloroquinolin-4-yl)-3-(difluoromethyl)pyridin-2-yl)oxy)-2,4-dimethylpentan-2-amine;
N-(4′-((2-amino-2,4-dimethylpentyl)oxy)-3′-methyl-[1,1′-biphenyl]-3-yl)acetamide;
(S)-1-(4-(2-(difluoromethyl)pyridin-4-yl)-2-(trifluoromethyl)phenoxy)-2,4-dimethylpentan-2-amine;
(S)-2,4-dimethyl-1-(4-(2-methylpyridin-4-yl)-2-(trifluoromethyl)phenoxy)pentan-2-amine;
(S)-methyl(5-((2-amino-2,4-dimethylpentyl)oxy)-6-methyl-[2,4′-bipyridin]-2′-yl)carbamate;
(S)-1-((4-(difluoromethyl)-2′-methyl-[2,4′-bipyridin]-5-yl)oxy)-2,4-dimethylpentan-2-amine;
(S)-1-((2′,6-bis(difluoromethyl)-[2,4′-bipyridin]-5-yl)oxy)-2,4-dimethylpentan-2-amine;
(S)-2,4-dimethyl-1-((2′-methyl-4-(trifluoromethyl)-[2,4′-bipyridin]-5-yl)oxy)pentan-2-amine;
(S)-1-((4-(difluoromethyl)-3′-fluoro-2′-methyl-[2,4′-bipyridin]-5-yl)oxy)-2,4-dimethylpentan-2-amine;
(S)-1-((6-(difluoromethyl)-3′-fluoro-2′-methyl-[2,4′-bipyridin]-5-yl)oxy)-2,4-dimethylpentan-2-amine;
(S)-1-((3-(difluoromethyl)-5-(2-methylpyrimidin-4-yl)pyridin-2-yl)oxy)-2,4-dimethylpentan-2-amine;
(S)-1-((4-chloro-2′-methyl-[2,4′-bipyridin]-5-yl)oxy)-2,4-dimethylpentan-2-amine; and pharmaceutically acceptable salts thereof.

A preferred AAK1 inhibitor is (S)-1-((2′,6-bis(difluoromethyl)-[2,4′-bipyridin]-5-yl)oxy)-2,4-dimethylpentan-2-amine:

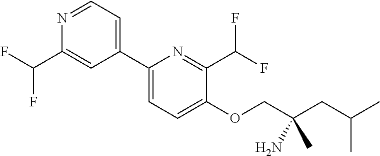 [SMILES: CC(C)C[C@](C)(N)COc1ccc(-c2ccnc(C(F)F)c2)nc1C(F)F]

or a pharmaceutically acceptable salt thereof. A specific salt is ((S)-1-((2′,6-bis(difluoromethyl)-[2,4′-bipyridin]-5-yl)oxy)-2,4-dimethylpentan-2-aminium dihydrogen phosphate.

The AAK1 inhibitors disclosed herein may exist in various solid (e.g., crystalline) forms. A particular solid form of ((S)-1-((2′,6-bis(difluoromethyl)-[2,4′-bipyridin]-5-yl)oxy)-2,4-dimethylpentan-2-aminium dihydrogen phosphate, referred to herein as Form I, is described in U.S. patent application publication US-2023-0357182-A1, filed Feb. 24, 2023. Crystalline Form I has an XRPD spectrum with diffraction peaks at one or more of about 4.81, 5.99, 7.44, 7.89, 11.66, 14.85, 15.77, 19.19, 20.86, 21.65, 23.96, 24.48, or 24.73 degrees 2-theta as measured using a Bruker X-ray diffractometer with a LYNXEYE detector (copper Kα radiation). When used herein to refer to XPRD peaks, the term “about” means±0.2 degrees 2-theta. Crystalline Form I has a melting point of about 184° C. as determined by differential scanning calorimetry (DSC) (melting endotherm). When referring to a temperature, the terms “substantially” and “about” mean±2° C.

The AAK1 inhibitors disclosed herein can be prepared by methods known in the art. See, e.g., U.S. Pat. No. 9,902,722; Luo G., et al., J Med Chem. 2022 Mar. 24;65(6):4534-4564; and Luo G, et al., J Med Chem. 2022 Mar. 24;65(6):4457-4480.

### Pharmaceutical Formulations and Kits

Pharmaceutical formulations comprising an AAK1 inhibitor may be adapted for administration by any appropriate route, for example by the oral (including buccal or sublingual), rectal, nasal, topical (including buccal, sublingual, or transdermal), vaginal, or parenteral (including subcutaneous, intracutaneous, intramuscular, intra-articular, intrasynovial, intrasternal, intrathecal, intralesional, intravenous, or intradermal injections or infusions) route. Such formulations may be prepared by any method known in the art of pharmacy, for example by bringing into association the active ingredient with the carrier(s) or excipient(s). Oral administration or administration by injection are preferred.

Pharmaceutical formulations adapted for oral administration may be presented as discrete units such as capsules or tablets; powders or granules; solutions or suspensions in aqueous or non-aqueous liquids; edible foams or whips; or oil-in-water liquid emulsions or water-in-oil emulsions.

For instance, for oral administration in the form of a tablet or capsule, the active drug component can be combined with an oral, non-toxic pharmaceutically acceptable inert carrier such as ethanol, glycerol, water, and the like. Powders are prepared by comminuting the compound to a suitable fine size and mixing with a similarly comminuted pharmaceutical carrier such as an edible carbohydrate, as, for example, starch or mannitol. Flavoring, preservative, dispersing, and coloring agent can also be present.

Capsules are made by preparing a powder mixture, as described above, and filling formed gelatin sheaths. Glidants and lubricants such as colloidal silica, talc, magnesium stearate, calcium stearate, or solid polyethylene glycol can be added to the powder mixture before the filling operation. A disintegrating or solubilizing agent such as agar-agar, calcium carbonate, or sodium carbonate can also be added to improve the availability of the medicament when the capsule is ingested.

Moreover, when desired or necessary, suitable binders, lubricants, disintegrating agents, and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, and the like. Lubricants used in these dosage forms include sodium oleate, sodium chloride, and the like. Disintegrators include, without limitation, starch, methyl cellulose, agar, betonite, xanthan gum, and the like. Tablets are formulated, for example, by preparing a powder mixture, granulating or slugging, adding a lubricant and disintegrant, and pressing into tablets. A powder mixture is prepared by mixing the compound, suitable comminuted, with a diluent or base as described above, and optionally, with a binder such as carboxymethylcellulose, an aliginate, gelating, or polyvinyl pyrrolidone, a solution retardant such as paraffin, a resorption accelerator such as a quaternary salt and/or and absorption agent such as betonite, kaolin, or dicalcium phosphate. The powder mixture can be granulated by wetting with a binder such as syrup, starch paste, acadia mucilage, or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc, or mineral oil. The lubricated mixture is then compressed into tablets. The compounds of the present disclosure can also be combined with a free-flowing inert carrier and compressed into tablets directly without going through the granulating or slugging steps. A clear or opaque protective coating consisting of a sealing coat of shellac, a coating of sugar or polymeric material, and a polish coating of wax can be provided. Dyestuffs can be added to these coatings to distinguish different unit dosages.

Oral fluids such as solution, syrups, and elixirs can be prepared in dosage unit form so that a given quantity contains a predetermined amount of the compound. Syrups can be prepared by dissolving the compound in a suitably flavored aqueous solution, while elixirs are prepared through the use of a non-toxic vehicle. Solubilizers and emulsifiers such as ethoxylated isostearyl alcohols and polyoxyethylene sorbitol ethers, preservatives, flavor additive such as peppermint oil or natural sweeteners, or saccharin or other artificial sweeteners, and the like can also be added.

Where appropriate, dosage unit formulations for oral administration can be microencapsulated. The formulation can also be prepared to prolong or sustain the release as for example by coating or embedding particulate material in polymers, wax, or the like.

The compounds of Formula (I), and pharmaceutically acceptable salts thereof, can also be administered in the form of liposome delivery systems, such as small unilamellar vesicles, large unilamellar vesicles, and multilamellar vesicles. Liposomes can be formed from a variety of phopholipids, such as cholesterol, stearylamine, or phophatidylcholines.

The compounds of Formula (I) and pharmaceutically acceptable salts thereof may also be delivered by the use of monoclonal antibodies as individual carriers to which the compound molecules are coupled. The compounds may also be coupled with soluble polymers as targetable drug carriers. Such polymers can include polyvinylpyrrolidone, pyran copolymer, polyhydroxypropylmethacrylamidephenol, polyhydroxyethylaspartamidephenol, or polyethyleneoxidepolylysine substituted with palitoyl residues. Furthermore, the compounds may be coupled to a class of biodegradable polymers useful in achieving controlled release of a drug, for example, polylactic acid, polepsilon caprolactone, polyhydroxy butyric acid, polyorthoesters, polyacetals, polydihydropyrans, polycyanoacrylates, and cross-linked or amphipathic block copolymers of hydrogels.

Pharmaceutical formulations adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research 1986, 3(6),318.

Pharmaceutical formulations adapted for topical administration may be formulated as ointments, creams, suspensions, lotions, powders, solutions, pastes, gels, sprays, aerosols, or oils.

Pharmaceutical formulations adapted for rectal administration may be presented as suppositories or as enemas.

Pharmaceutical formulations adapted for nasal administration wherein the carrier is a solid include a course powder having a particle size for example in the range 20 to 500 microns which is administered in the manner in which snuff is taken, i.e., by rapid inhalation through the nasal passage from a container of the powder held close up to the nose. Suitable formulations wherein the carrier is a liquid, for administration as a nasal spray or nasal drops, include aqueous or oil solutions of the active ingredient.

Pharmaceutical formulations adapted for administration by inhalation include fine particle dusts or mists, which may be generated by means of various types of metered, dose pressurized aerosols, nebulizers, or insufflators.

Pharmaceutical formulations adapted for vaginal administration may be presented as pessaries, tampons, creams, gels, pastes, foams, or spray formulations.

Pharmaceutical formulations adapted for parenteral administration include aqueous and non-aqueous sterile injection solutions which may contain antioxidants, buffers, bacteriostats, and soutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules, and tablets.

It should be understood that in addition to the ingredients particularly mentioned above, the formulations may include other agents conventional in the art having regard to the type of formulation in question, for example those suitable for oral administration may include flavoring agents.

This invention also encompasses kits comprising one or more pharmaceutical compositions that may be used to practice methods disclosed herein. Some kits comprise an AAK1 inhibitor (e.g., a compound of Formula I) and at least one chemotherapeutic drug. Some kits further comprise one or more additional therapeutic agents that may alleviate one or more symptoms of CINP. Examples of such therapeutics include selective serotonin and norepinephrine reuptake inhibitors (e.g., duloxetine), venlafaxine, pregabalin, and carbamazepine.

A kit may further comprise buffers and other compositions to aid in administration to a patient in need of treatment. The compositions may be formulated for specific dosing regimens. Each composition or solution may be contained in a vial or bottle and all components included in a box for commercial sale. The pharmaceutical compositions can be included in a container, pack, or dispensed together with instructional materials. Particular kits further comprise buffers, containers, and instructional materials (e.g., regarding the preparation and administration of the drugs, safety information, and the like).

### Methods of Use

This invention is directed, in part, to methods of treating, managing, and preventing CINP in a patient suffering from the condition. Particular embodiments of the invention comprise inhibiting AAK1. In preferred embodiments, the AAK1 inhibition is achieved by administering to the patient an effective amount of an AAK1 inhibitor. Preferred AAK1 inhibitors penetrate the central nervous system, and are disclosed herein. A particularly preferred AAK1 inhibitor is (S)-1-((2′,6-bis(difluoromethyl)-[2,4′-bipyridin]-5-yl)oxy)-2,4-dimethylpentan-2-amine or a pharmaceutically acceptable salt thereof.

Particular embodiments of the invention comprise methods of treating, managing, or preventing CINP in a patient in need thereof, which comprise administering a therapeutically or prophylactically effective amount of an AAK1 inhibitor to the patient. Patients in need of such treatment include those undergoing chemotherapy (e.g., patients with acute CINP) and patients that have completed chemotherapy but whose CINP symptoms remain (e.g., patients with chronic CINP). Patients in need of such prevention include patients who are about to undergo chemotherapy.

Particular patients have or did have cancer. Some specific cancers that are commonly treated with drugs that can cause CINP include colorectal cancer, neuroblastoma, biliary adenocarcinoma, leukemia, esophageal cancer, gastric cancer, neuroendocrine tumors, non-Hodgkin lymphoma, pancreatic cancer, ovarian cancer, and testicular cancer.

In certain embodiments, the AAK1 inhibitor is administered in combination—albeit not necessarily at the same time or in the same pharmaceutical formulation—with one or more chemotherapeutic agents. Examples of chemotherapeutic agents include chelated platinum, taxanes, proteosome inhibitors, vinca alkaloids, and immunomodulatory drugs. Specific examples include 5-fluorouracil, capecitabine, cisplatin, cytarabine, docetaxel, doxorubicin, epirubicin, etoposide, fludarabine, gemcitabine, ifosfamide, irinotecan, leucovorin, oxaliplatin, paclitaxel, and rituximab.

In certain embodiments, the AAK1 inhibitor is administered in combination—albeit not necessarily at the same time or in the same pharmaceutical formulation—with another therapeutic agent that may alleviate one or more symptoms of CINP. Examples of such therapeutics include selective serotonin and norepinephrine reuptake inhibitors (e.g., duloxetine), venlafaxine, pregabalin, and carbamazepine.

An AAK1 inhibitor can be administered to a subject in one or more doses. In some embodiments, the AAK1 inhibitor can be administered in an amount of about 10 mg to 1000 mg per dose, e.g., about 10 mg to 20 mg, about 20 mg to 25 mg, about 25 mg to 50 mg, about 50 mg to 75 mg, about 75 mg to 100 mg, about 100 mg to 125 mg, about 125 mg to 150 mg, about 150 mg to 175 mg, about 175 mg to 200 mg, about 200 mg to 225 mg, about 225 mg to 250 mg, about 250 mg to 300 mg, about 300 mg to 350 mg, about 350 mg to 400 mg, about 400 mg to 450 mg, about 450 mg to 500 mg, about 500 mg to 750 mg, or about 750 mg to 1000 mg per dose. In some embodiments, the AAK1 inhibitor is administered to the subject in one or more single dose(s) of about 40 mg. In some embodiments, the AAK1 inhibitor is administered to the subject in one or more single dose(s) of about 200 mg.

In some embodiments, the amount of the AAK1 inhibitor per dose is determined on a per body weight basis. For example, in some embodiments, the AAK1 inhibitor can be administered in an amount of about 0.5 mg/kg to 100 mg/kg, e.g., about 0.5 mg/kg to 1 mg/kg, about 1 mg/kg to 2 mg/kg, about 2 mg/kg to 3 mg/kg, about 3 mg/kg to 5 mg/kg, about 5 mg/kg to 7 mg/kg, about 7 mg/kg to about 10 mg/kg, about 10 mg/kg to 15 mg/kg, about 15 mg/kg to 20 mg/kg, about 20 mg/kg to 25 mg/kg, about 25 mg/kg to 30 mg/kg, about 30 mg/kg to 40 mg/kg, about 40 mg/kg to 50 mg/kg, about 50 mg/kg to 60 mg/kg, about 60 mg/kg to 70 mg/kg, about 70 mg/kg to 80 mg/kg, about 80 mg/kg to 90 mg/kg, or about 90 mg/kg to 100 mg/kg, or more than about 100 mg/kg.

Those of skill will readily appreciate that dose levels can vary as a function of several different factors including, without limitation, the specific AAK1 inhibitor administered, the severity of the symptoms, the age and/or physical size of the subject, and the susceptibility of the subject to side effects. Preferred dosages for a given compound are readily determinable by those of skill in the art by a variety of means.

In some embodiments, multiple doses of the AAK1 inhibitor are administered. The frequency of administration of the AAK1 inhibitor can vary depending on any of a variety of factors, e.g., severity of the symptoms, and the like. For example, in some embodiments, the AAK1 inhibitor is administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid). As discussed above, in some embodiments, the AAK1 inhibitor is administered continuously.

To prevent CINP, an AAK1 inhibitor may be administered prior to (e.g., within 1, 2, 4, 6, 12, 24, or 48 hours before) the administration of the chemotherapeutic agent, during a chemotherapy session (e.g., concurrently with administration of the agent), or shortly thereafter (e.g., within 1, 2, 4, 6, 12, or 24 hours following) the administration of the chemotherapeutic agent. Treatment of CINP may occur any time after a patient exhibits symptoms.

The duration of administration of the AAK1 inhibitor is administered, can vary, depending on any of a variety of factors known by those skilled in the art (e.g., patient response, route of administration, dosage form). For example, the AAK1 inhibitor can be administered over a period of time of about one day to one week, about two weeks to four weeks, about one month to two months, about two months to four months, about four months to six months, about six months to eight months, about eight months to 1 year, about 1 year to 2 years, or more.

Embodiments of the present invention provide methods and compositions for the administration of the AAK1 inhibitor to a patient (e.g., a human) using any available method and route suitable for drug delivery, including in vivo and ex vivo methods, as well as systemic and localized routes of administration.

Routes of administration include oral, intranasal, intramuscular, intratracheal, subcutaneous, intradermal, topical application, intravenous, rectal, nasal, and other enteral and parenteral routes of administration. Routes of administration may be combined, if desired, or adjusted depending upon the agent and/or the desired effect. An active agent can be administered in a single dose or in multiple doses.

Embodiments of the AAK1 inhibitor can be administered to a host using available conventional methods and routes suitable for delivery of conventional drugs, including systemic or localized routes. In general, routes of administration contemplated by the disclosure include, but are not limited to, enteral, parenteral, or inhalational routes.

Parenteral routes of administration other than inhalation administration include, but are not limited to, topical, transdermal, subcutaneous, intramuscular, intraorbital, intracapsular, intraspinal, intrasternal, and intravenous routes. Parenteral administration can be conducted to effect systemic or local delivery of the AAK1 inhibitor. Where systemic delivery is desired, administration typically involves invasive or systemically absorbed topical or mucosal administration of pharmaceutical preparations.

A preferred method of administering Compound A is oral, once daily, at a dose of from about 3 mg/kg to 30 mg/kg. In one method, Compound A is first administered to a human patient with a 100 mg loading dose, followed by 10 mg maintenance doses thereafter. In another, Compound A is first administered to a human patient with a 200 mg loading dose, followed by 20 mg maintenance doses thereafter.

### EXAMPLES

Aspects of particular embodiments of this invention are illuminated by the following examples.

### Example 1. Tablet Dosage Forms

Tablets comprising 10, 20, or 50 mg of Compound A as a dihydrogen phosphate salt were prepared in a batch of 20,000 using the ingredients shown below:

TABLE 1

| | Amount (mg) Per | | |
|---|---|---|---|
| | 10 mg | 20 mg | 50 mg |
| Ingredient | batch | batch | batch |
| Compound A^{1, 2} | 252.8 | 505.6 | 1264.0 |
| Silicified Microcrystalline Cellulose | 2758.6 | 2632.2 | 2253.0 |
| Dibasic Calcium Phosphate, Anhydrous | 2758.6 | 2632.2 | 2253.0 |
| Croscarmellose Sodium | 120.0 | 120.0 | 120.0 |
| Talc | 120.0 | 120.0 | 120.0 |
| Colloidal Silicon Dioxide | 30.0 | 30.0 | 30.0 |
| Hydrogenated Vegetable Oil | 160.0 | 160.0 | 160.0 |
| Total (Core Tablet) | 6200.0 | 6200.0 | 6200.0 |
| OpaDry II White³ | 248.0 | 248.0 | 248.0 |
| Total (Film coated Tablet) | 6448.0 | 6448.0 | 6448.0 |

¹One mg of Compound A is equivalent to 0.7973 mg of the corresponding free base.
²The quantity of Compound A is adjusted for potency based on the supplier Certificate of Analysis, Assay (Weight %, HPLC). A proportionate quantity of Silicified Microcrystalline Cellulose and Dibasic Calcium Phosphate, Anhydrous is adjusted accordingly.
³Opadry II White is dispersed in Purified Water at 15% solids to coat the cores. A 50% overage of the coating is prepared to compensate for losses during film-coating and to guarantee the amount applied per tablet. Purified Water is removed during production.

The following steps were followed to prepare solid oral dosage forms of Compound A:

1. The quantity of Compound A is adjusted for potency based on the supplier Certificate of Analysis, Assay (Weight %, HPLC).

2. Screen Compound A, Silicified Microcrystalline Cellulose, Dibasic Calcium Phosphate, Anhydrous, Croscarmellose Sodium into a bin blender and blend. Note: A proportionate quantity of Silicified Microcrystalline Cellulose and Dibasic Calcium Phosphate, Anhydrous is adjusted according to the Compound A potency, as determined in Step 1.

3. Screen Talc and Colloidal Silicon Dioxide into the blend from Step 2 and blend.

4. Screen Hydrogenated Vegetable Oil into the blend from Step 3 and blend.

5. Compress the blend from Step 4.

6. The tablet cores, from Step 5, are subsequently coated with an aqueous dispersion of Opadry II White for an approximate weight gain of 4%.

7. The coated tablets, from Step 6, are packaged into HDPE bottles induction sealed with a child resistant cap.

### Example 2. Rat Model of Oxaliplatin-Induced Peripheral Neuropathy

Oxaliplatin is a third-generation platinum-based chemotherapy drug widely used in the treatment of advanced metastatic colorectal cancer. This experimental model of oxaliplatin-induced peripheral sensory neuropathy is induced by repeated intraperitoneal injections of oxaliplatin (4 mg/kg) two times per week for three weeks. Cold allodynia is measured using the cryothermostat. See Singh, V. P., Kulkarni, S. K., “Effect of licofelone against mechanical hyperalgesia and cold allodynia in the rat model of incisional pain” Pharmacological Reports 2005(57):380-384. This model displays significant behavioral nociceptive signs and is consistent with clinical symptoms that increase progressively in duration and severity with the repetition of treatment-cycles. Cheng, X., et al., “Herbal Medicine AC591 Prevents oxaliplatin-Induced Peripheral Neuropathy in Animal Model and Cancer Patients” Frontiers in Pharmacology, 2017(8):344. This model is useful to evaluate the analgesic effects of new compounds on oxaliplatin-induced chronic painful symptoms.

This implementation of the model used sixty (60) male Sprague-Dawley rats, weighing 103-139 g during the first oxaliplatin injection were used. The rats were housed in a temperature (20-24° C.) and relative humidity (45%-65%) controlled room and acclimated to an artificial day/night cycle of 12 hours light (6.30 a.m. to 6.30 p.m.)/12 hours darkness. Rats had free access to tap water and were fed ad libitum with pelleted complete diet. Animals were housed 3 or 2 per cage and were acclimated for a period of at least 5 days before any testing. Each rat was identified by tail markings.

Cold allodynia was measured using the paw immersion test. See Singh, supra. In this test, the latency of hindpaw withdrawal was measured after immersion of the hindpaw in the cryothermostat with a temperature fixed at 10° C. (±0.5° C.).

### Example 3. Assessment of Single Administration of Compound

This study sought to determine the efficacy of a single administration of ((S)-1-((2′,6-bis (difluoromethyl)-[2,4′-bipyridin]-5-yl)oxy)-2,4-dimethylpentan-2-aminium dihydrogen phosphate (“Compound A”) at two doses in the model of oxaliplatin-induced peripheral neuropathy (cold allodynia) described above in Example 2. Duloxetine was used as a reference to validate the assay.

Sixty (60) male Sprague-Dawley rats, weighing 103-139 g during the first oxaliplatin injection were used. Chronic peripheral neuropathy was induced by repeated intraperitoneal injections of oxaliplatin (4 mg/kg, i.p., 10 ml/kg) two times a week for three weeks (CD=24 mg/kg, i.p.). Four experimental groups were treated with oxaliplatin (4 mg/kg, i.p.) and one (group 1) was treated with 5% glucose (vehicle of oxaliplatin) following the same sequence of administration.

On day zero (D0), latency of hindpaw withdrawal was measured on both paws using the cryothermostat (Baseline pre-induction). On the first week, two intraperitoneal injections of oxaliplatin 4 mg/kg (group 2, 3, 4 and 5) or 5% glucose (group 1) were performed (D0 and D3). On D4, the hindpaw withdrawal latency was measured on both paws using a cryothermostat to obtain a postinduction baseline. Rats were then treated with vehicle, Compound A, or duloxetine (T0). Sixty minutes later, the latency of hindpaw withdrawal was measured on both paws using the cryothermostat. The same sequence of oxaliplatin injections, administration of compounds, and test was performed in the second week (D7, D10, D11) and in the third week (D14, D17, D18).

Intraperitoneal administrations of oxaliplatin 4 mg/kg twice a week induced a marked and significant decrease of paw withdrawal latency three weeks after the first injection for all groups (except for sham animals). Rats from the vehicle-treated group exhibited a reduced nociceptive latency reaction throughout the three weeks. Duloxetine administrated at 100 mg/kg showed significant increase in paw withdrawal latency as compared to the vehicle-treated group at weeks 2 and 3. Compound A administered at 30 mg/kg induced a slight but significant increase in the paw withdrawal latency at week 3, 60 minutes post-treatment as compared to the vehicle-treated group.

Five (5) experimental groups of 12 rats each were used:
Group 1: 5% Glucose+10% Ethanol/40% PEG 400/15% Tween 80 in sterile water, p.o., solution.
Group 2: Oxaliplatin 4 mg/kg 3×(TW)+10% Ethanol/40% PEG 400/15% Tween 80 in sterile water, p.o., solution.
Group 3: Oxaliplatin 4 mg/kg 3×(TW)+Compound A (10 mg/kg, p.o.) in vehicle, suspension.
Group 4: Oxaliplatin 4 mg/kg 3×(TW)+Compound A (30 mg/kg, p.o.) in vehicle, suspension.
Group 5: Oxaliplatin 4 mg/kg 3×(TW)+Duloxetine (100 mg/kg, p.o.) in 1% MC, suspension.

Vehicle (10% Ethanol/40% PEG 400/15% Tween 80 in sterile water) and Compound A were orally administered at 3 ml/kg. Duloxetine was orally administered at 10 ml/kg. Dosing and testing were performed in a random order by a blinded experimenter except for sham animals and duloxetine-treated group. Doses are expressed in terms of free active substance.

Peripheral neuropathy was induced by repeated intraperitoneal injections of oxaliplatin (4 mg/kg, i.p., 10 ml/kg) two times a week for three weeks (CD=24 mg/kg, i.p.). Four experimental groups were treated with oxaliplatin (4 mg/kg, i.p.) and one (group 1) was treated with 5% glucose (vehicle for oxaliplatin) following the same sequence of administration.

On D0, latency of hindpaw withdrawal was measured on both paws using the cryothermostat to obtain a preinduction baseline preinduction. In the first week, two intraperitoneal injections of oxaliplatin 4 mg/kg (group 2, 3, 4 and 5) or 5% glucose (group 1) were performed (D0 and D3). On D4, the hindpaw withdrawal latency was measured on both paws using the cryothermostat (post-induction baseline). Then, rats were treated with vehicle, Compound A, or duloxetine at T0. Sixty minutes later, the latency of hindpaw withdrawal was measured on both paws using the cryothermostat. The same sequence of oxaliplatin injections, administration of compounds, and test was performed in the second week (days D7, D10, D11) and in the third week (days D14, D17, D18). Rats were sacrificed at the end of the experiment by CO₂ inhalation pending subsequent removal by a certified company.

Results were expressed as: 1) the paw withdrawal latency (mean±s.e.m.) in seconds for each group, calculated from individual paw withdrawal latency; and 2) the percentage of variation of the paw withdrawal latency calculated from the mean value of the vehicle-treated group. To determine a statistical effect of the test substance and the reference substance, data were analyzed by a non-parametrical test. The significance level was p<0.05.

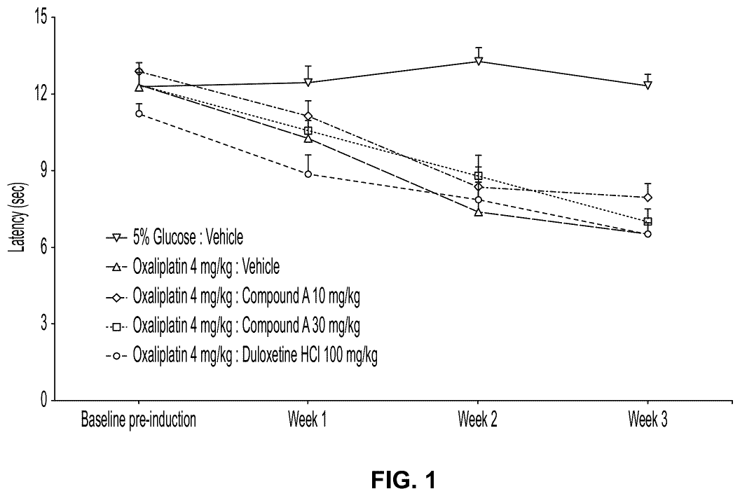

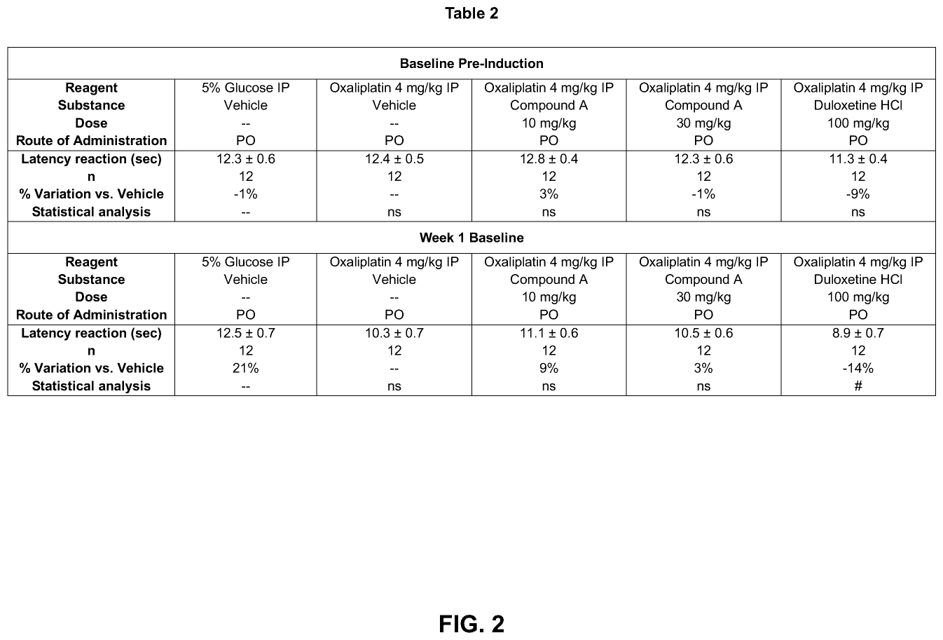

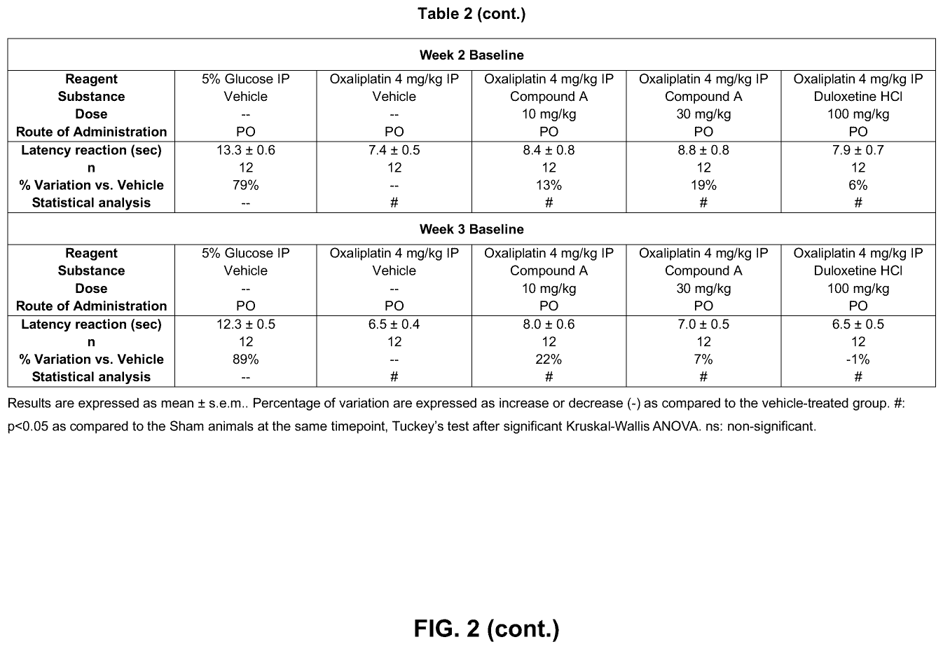

As shown in FIG. 1 and Table 2 (FIG. 2), intraperitoneal administrations of oxaliplatin 4 mg/kg twice a week induced a marked and significant decrease of paw withdrawal latency three weeks after the first injection for all groups (except for sham animals) as compared to the sham animals at the same time point.

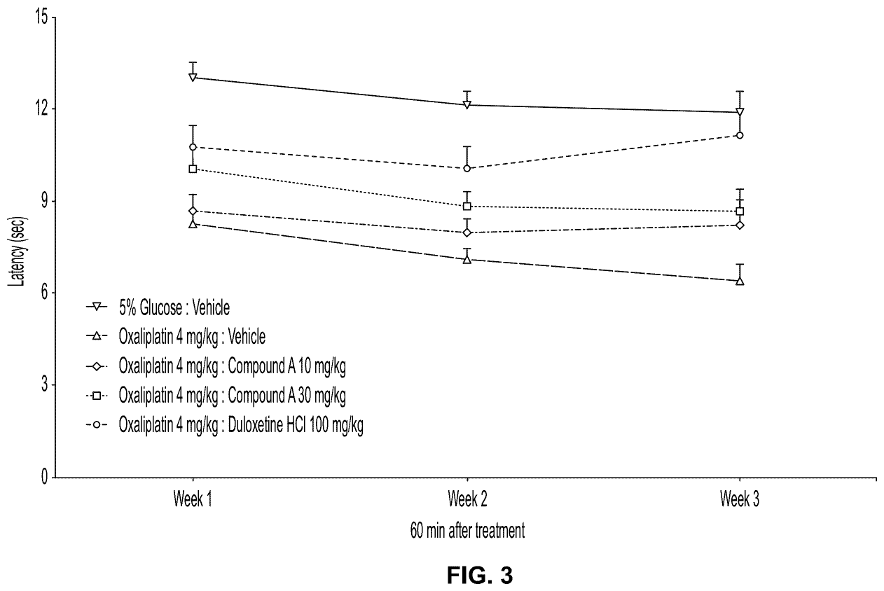

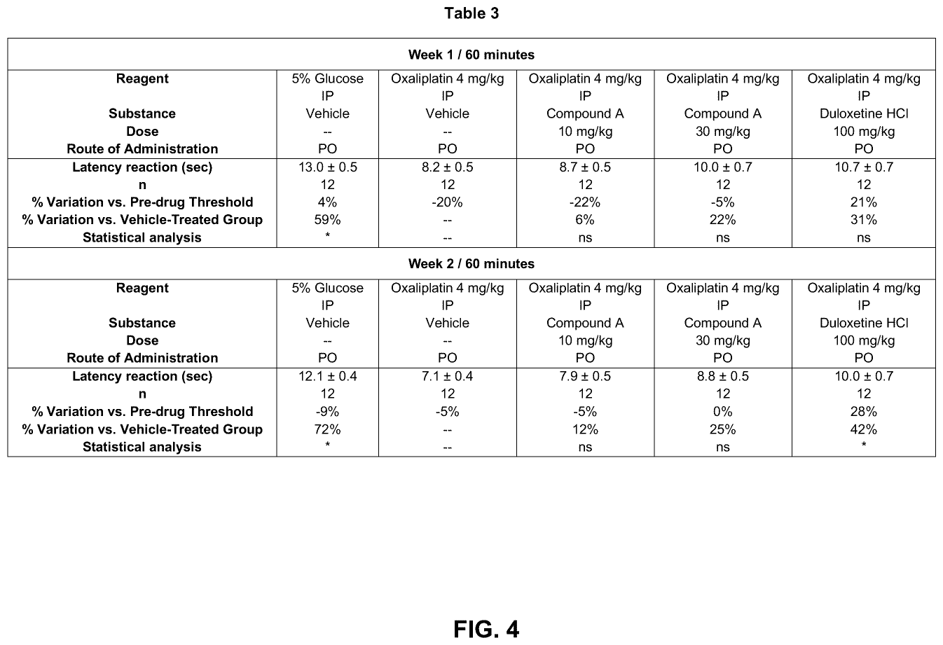

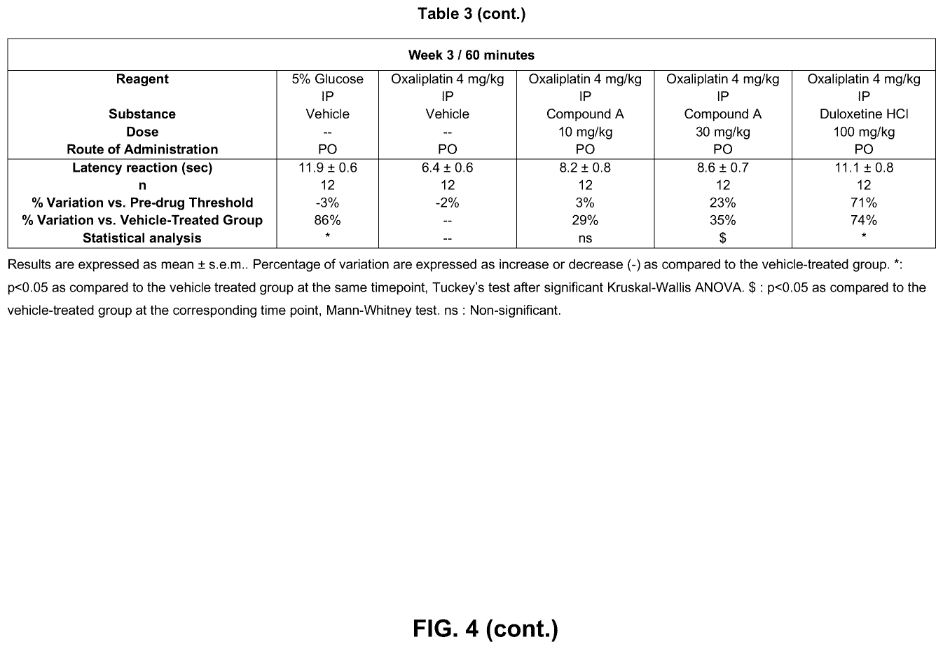

As shown in FIG. 3 and Table 3 (FIG. 4), rats from the vehicle-treated group exhibited a reduced nociceptive latency reaction throughout the three weeks, with the lowest at week 3 (6.4±0.6 sec). Duloxetine administrated at 100 mg/kg showed significant increase in the paw withdrawal latency as compared to the vehicle-treated group at week 2 and week 3 (+42%, +74% respectively; p<0.05). Compound A administered at 10 mg/kg did not show a statistically significant increase in paw withdrawal latency as compared to the vehicle-treated group. However, when administered at 30 mg/kg, Compound A induced a statistically significant increase in the paw withdrawal latency at week 3, 60 minutes post-treatment as compared to the vehicle-treated group (+35%, p<0.05).

### Example 4. Assessment of Repeated Administration of Compound

This study sought to determine the efficacy of repeated administration of Compound A at two doses in the model of oxaliplatin-induced peripheral neuropathy (cold allodynia) described above in Example 2. Duloxetine was used as a reference to validate the assay.

Sixty (60) male Sprague-Dawley rats, weighing 95-135 g during the first oxaliplatin injection were used. Peripheral neuropathy was induced by repeated intraperitoneal injections of oxaliplatin (4 mg/kg, i.p., 10 ml/kg) 2 times a week over three weeks (CD=24 mg/kg, i.p.). Four experimental groups were treated with oxaliplatin (4 mg/kg, i.p.) and one (group 1) was treated with 5% glucose (vehicle of oxaliplatin) following the same sequence of administration.

On D-3, latency of hindpaw withdrawal was measured on both paws using the cryothermostat to obtain a pre-induction and pre-treatment baseline. In the first week, two intraperitoneal injections of oxaliplatin 4 mg/kg (groups 2, 3, 4, and 5) or 5% glucose (group 1) were performed on days zero and three (D0, D3). The same sequence of oxaliplatin injections was performed in the second week (days D7, D10) and in the third week (days D14, D17). The first administration of vehicle and Compound A (groups 1 to 4) was done on D-3, then vehicle and Compound A were daily orally administered until D18 (n=22 repeated treatments). The animals treated with Compound A at 30 mg/kg had a dosing holiday on D8, and from D9 until D18, these animals were daily orally administered at 10 mg/kg instead of 30 mg/kg. On the testing days (D4, D11, and D18), latency of hindpaw withdrawal was measured on both paws using the cryothermostat to obtain a baseline. Duloxetine was then orally administered at the same time as vehicle and Compound A at time T0. Sixty minutes later, the latency of hindpaw withdrawal was measured.

Intraperitoneal administrations of oxaliplatin 4 mg/kg twice a week induced a marked and significant decrease of paw withdrawal latency two and three weeks after the first injection (with the exception of the sham animals and the 10 mg/kg Compound A group) as compared to the sham animals at the same time point. Rats from the vehicle-treated group exhibited a reduced nociceptive latency reaction in weeks 2 and 3. Duloxetine administrated at 100 mg/kg showed significant increase in paw withdrawal latency as compared to the vehicle-treated group at week 2 (+33%; p<0.01). In week 3, the duloxetine-treated animals presented an increase in the paw withdrawal latency as compared to the pre-drug baseline of the corresponding group (+50%; not significant (ns or NS)). These results validated the study.

Compound A daily administered at 30 mg/kg then 10 mg/kg presented a marked increase of the paw withdrawal latency at weeks 2 and 3 as compared to the vehicle-treated group. At week 3, 60 minutes after the administration, Compound A at 10 mg/kg induced a significant increase in the paw withdrawal latency as compared to the vehicle-treated group. Daily oral administrations of Compound A at 3 mg/kg did not show a statistically significant increase in the paw withdrawal latency as compared to the vehicle-treated group.

Five (5) experimental groups of 12 rats each were used:
Group 1: 5% Glucose+10% Ethanol/40% PEG 400/15% Tween 80 in sterile water, repeated administrations, p.o., solution.
Group 2: Oxaliplatin 4 mg/kg 3×(TW)+10% Ethanol/40% PEG 400/15% Tween 80 in sterile water, repeated administrations, p.o., solution.
Group 3: Oxaliplatin 4 mg/kg 3×(TW)+Compound A (3 mg/kg, p.o.) in vehicle, repeated administrations, suspension.
Group 4: Oxaliplatin 4 mg/kg 3×(TW)+Compound A (30 mg/kg, p.o.) in vehicle, repeated administrations, suspension.
Group 5: Oxaliplatin 4 mg/kg 3×(TW)+Duloxetine (100 mg/kg, p.o.) in 1% MC, suspension.

Vehicle (10% Ethanol/40% PEG 400/15% Tween 80 in sterile water) and Compound A were orally administered at 3 ml/kg. Duloxetine was orally administered at 10 ml/kg. Dosing and testing were performed in a random order by a blinded experimenter with the exception of sham animals and the duloxetine-treated group. Doses are expressed in terms of free active substance.

Chronic peripheral neuropathy was induced by repeated intraperitoneal injections of oxaliplatin (4 mg/kg, i.p., 10 ml/kg) 2 times a week over 3 weeks (CD=24 mg/kg, i.p.). Four experimental groups were treated with oxaliplatin (4 mg/kg, i.p.) and one (group 1) was treated with 5% glucose (vehicle of oxaliplatin) following the same sequence of administration.

On D-3, the latency of hindpaw withdrawal was measured on both paws using the cryothermostat to obtain preinduction and pretreatment baselines.

In the first week, two intraperitoneal injections of oxaliplatin 4 mg/kg (group 2, 3, 4 and 5) or 5% glucose (group 1) were performed (D0, D3). The same sequence of oxaliplatin injections was performed in the second week (D7, D10) and in the third week (D14, D17). The first administration of vehicle and Compound A (group 1 to 4) was done on D-3 then vehicle and Compound A were daily orally administered until D18 (n=22 repeated treatments). The animals treated with Compound A at 30 mg/kg had a dosing holiday on D8, and from D9 these animals were daily orally administered at 10 mg/kg instead of 30 mg/kg, until D18.

On the testing days (D4, D11 and D18), latency of hindpaw withdrawal was measured on both paws using the cryothermostat (baseline) then duloxetine was orally administered at the same time than vehicle and Compound A (T0) and 60 minutes later, the latency of hindpaw withdrawal was measured.

Rats were sacrificed at the end of the experiment by CO₂ inhalation pending subsequent removal by a certified company.

Results were expressed as: 1) the paw withdrawal latency (mean±s.e.m.) in seconds for each group, calculated from individual paw withdrawal latency; and 2) the percentage of variation of the paw withdrawal latency calculated from the mean value of the vehicle-treated group. To determine a statistical effect of the test substance and the reference substance, data were analyzed by a non-parametrical test. The significance level was p<0.05.

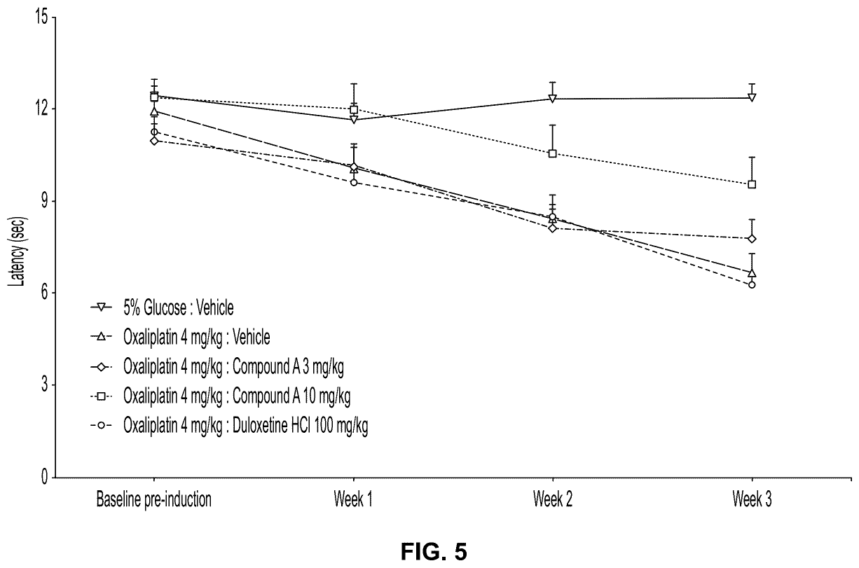

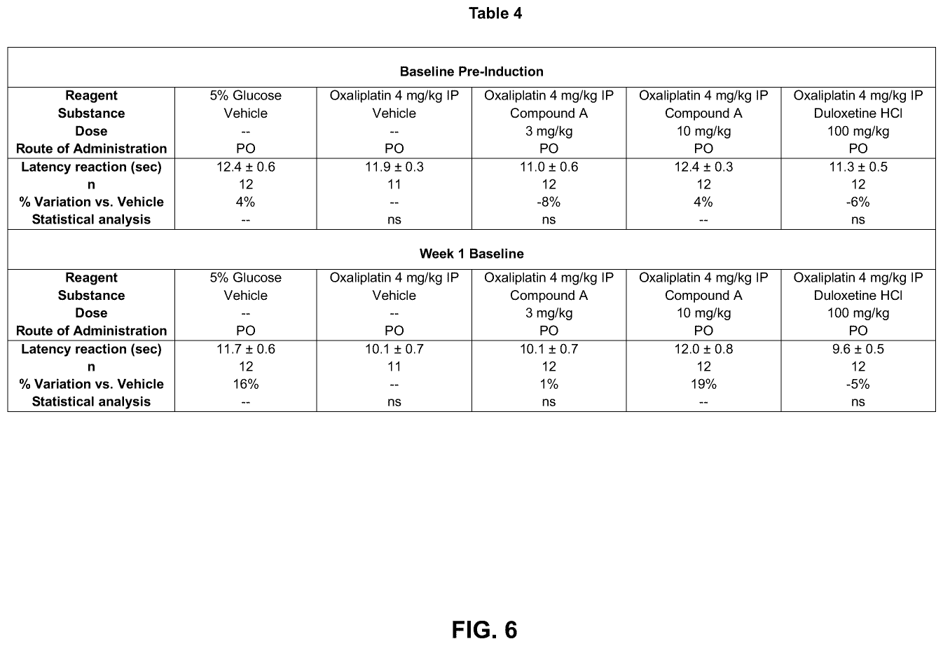

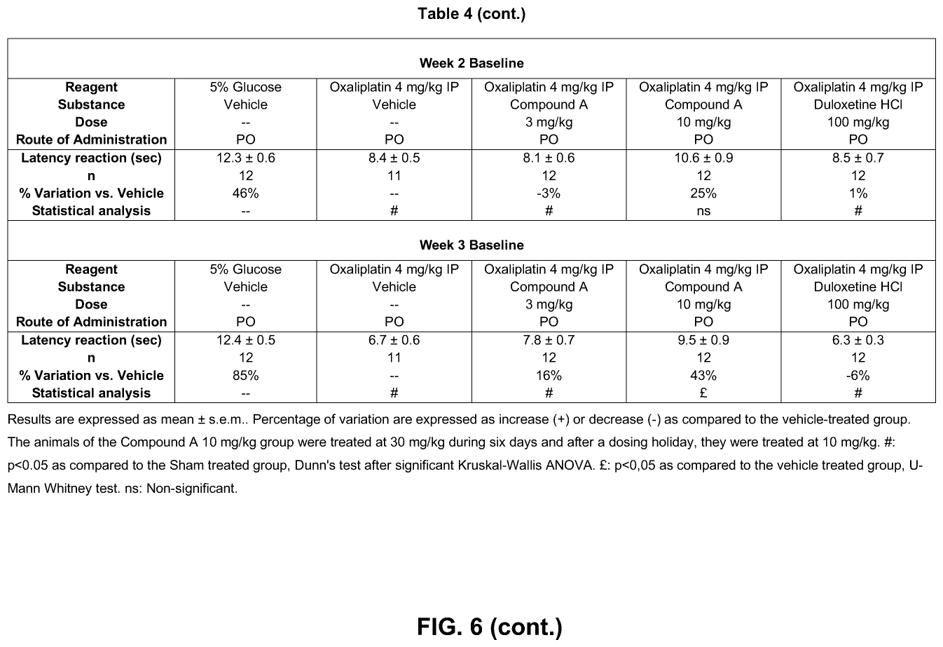

As presented in FIG. 5 and Table 4 (FIG. 6), intraperitoneal administration of oxaliplatin 4 mg/kg twice a week induced a marked and significant decrease of paw withdrawal latency two and three weeks after the first injection, for three groups (vehicle, Compound A 3 mg/kg, and duloxetine) as compared to the sham animals at the same time point.

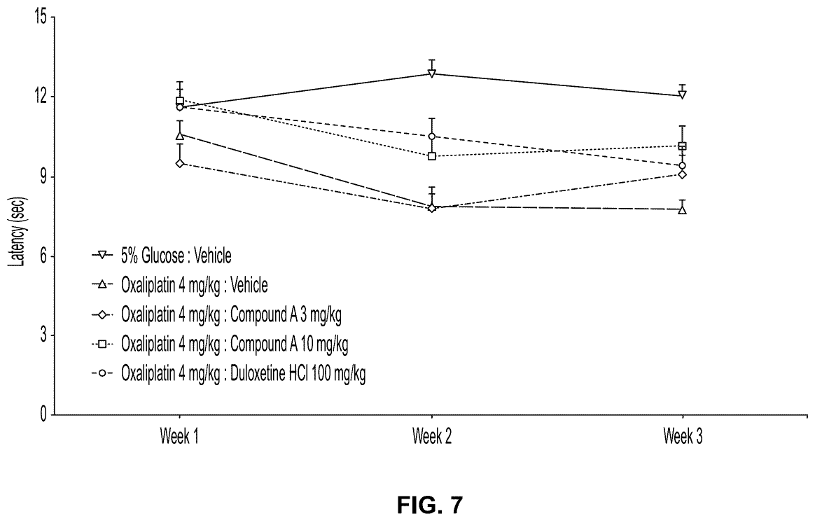

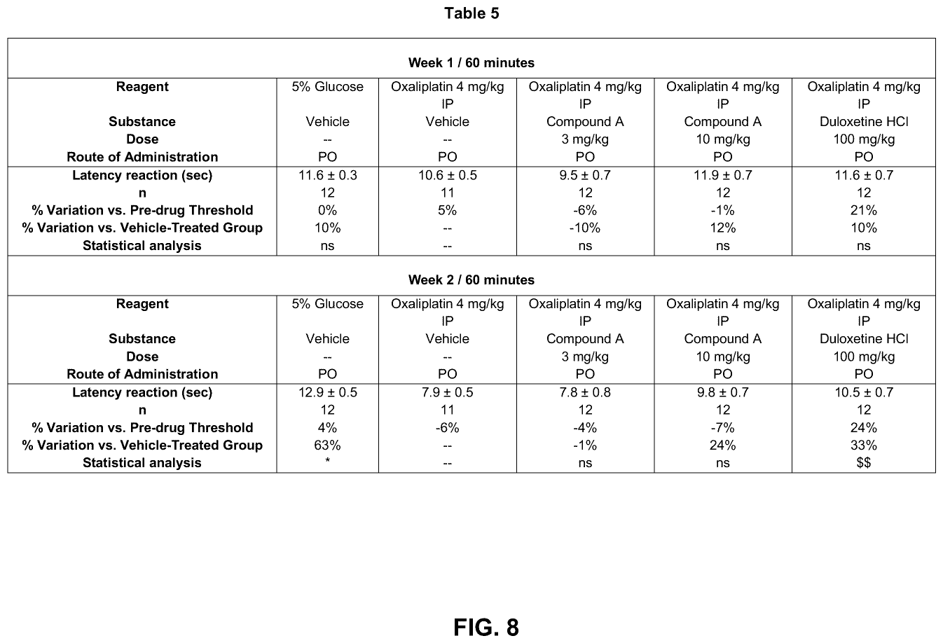

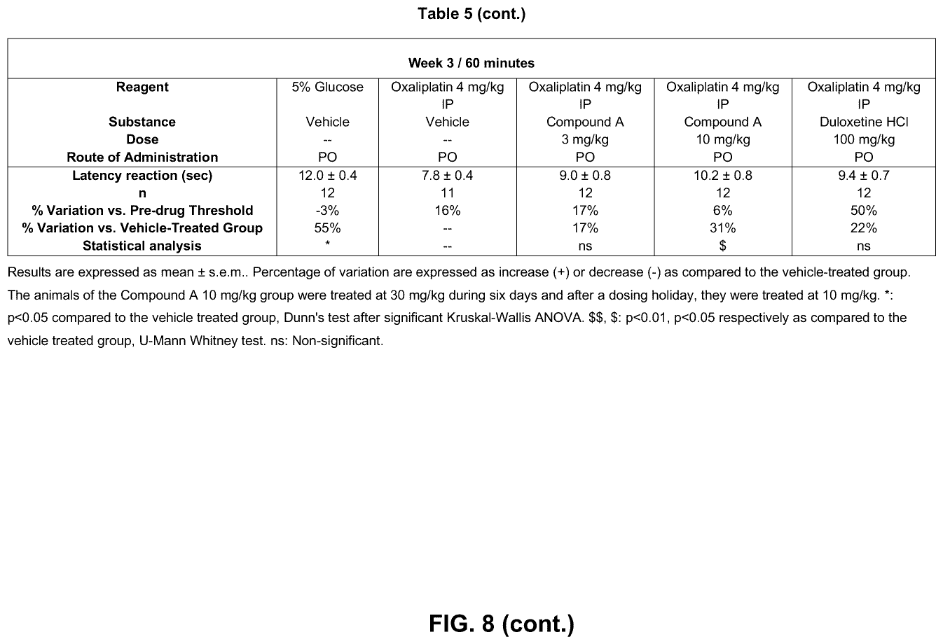

As showed on FIG. 7 and Table 5 (FIG. 8), rats from the vehicle-treated group exhibited a reduced nociceptive latency reaction on weeks 2 and 3, with the lowest at week 3 (7.8±0.4 sec).

Duloxetine administrated at 100 mg/kg showed significant increase in the paw withdrawal latency as compared to the vehicle-treated group at week 2 (+33%; p<0.01). At week 3, these animals presented an increase in the paw withdrawal latency as compared to the pre-drug baseline of the corresponding group (9.4±0.7 sec vs 6.3±0.3 sec; +50%; ns). These results validated the study.

As showed in FIG. 5 and Table 4, Compound A daily administered at 30 mg/kg then 10 mg/kg presented a marked increase of the paw withdrawal latency on week 2 (+25%; ns) and week 3 (+43%; p<0.05) as compared to the vehicle-treated group. As showed on FIG. 7 and Table 5 (FIG. 8), at week 3, 60 minutes after the administration, Compound A 10 mg/kg induced a significant increase in the paw withdrawal latency as compared to the vehicle-treated group (10.2±0.8 sec vs 6.7±0.6 sec; p<0.05).

All publications (e.g., patents and patent applications) cited above are incorporated herein by reference in their entireties.

1. A method of treating, managing, or preventing chemotherapy-induced peripheral neuropathy (CIPN), which comprises administering a therapeutically or prophylactically effective amount of an adaptor associated kinase 1 (AAK1) inhibitor to a patient in need thereof, wherein the AAK1 inhibitor is a compound of Formula I: 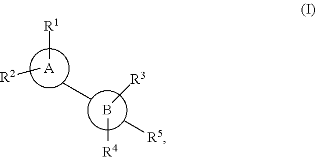
or a pharmaceutically acceptable salt thereof, wherein:
A is selected from 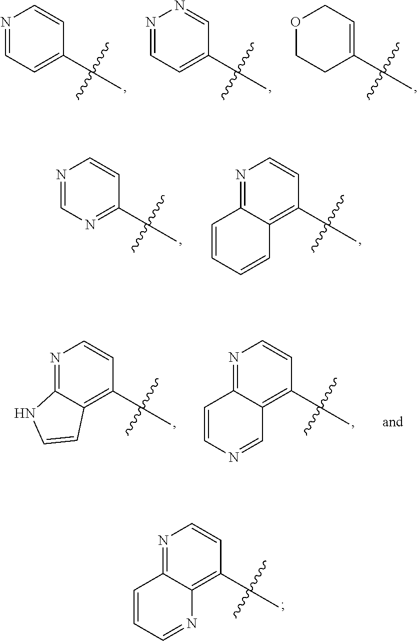
wherein “ [symbol] ” denotes the point of attachment to B;
B is selected from 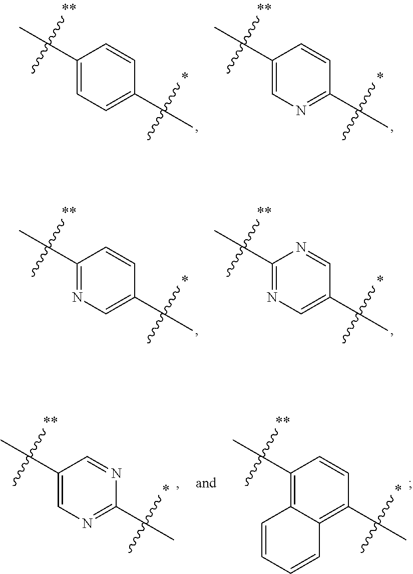 [SMILES: *c1ccc(*)cc1 ; *c1ccc(*)nc1 ; *c1ccc(*)nc1 ; *c1cnc(*)nc1 ; *c1cnc(*)nc1 ; *c1ccc(*)c2ccccc12]
wherein “*” indicates the point of attachment to R⁵ and “**” indicates the point of attachment to ring A;
R¹ is selected from hydrogen, amino, —CO₂H, difluoromethyl, ethyl, halo, hydroxymethyl, methoxy, methyl, —NHC(O)CH₃, —NHCO₂CH₃, trifluoromethoxy, and trifluoromethyl;
R² is selected from hydrogen, cyano, —CH₂OH, halo, and methyl;
R³ is selected from hydrogen, cyano, cyclopropyl, difluoromethyl, halo, hydroxymethyl, methoxy, methyl, methylsulfonyl, trifluoromethoxy, trifluoromethyl, —CH₂N(CH₃)₂, and a five-membered aromatic ring containing one, two, or three heteroatoms selected from nitrogen, oxygen, and sulfur;
R⁴ is selected from hydrogen, halo, and methyl;
R⁵ is selected from 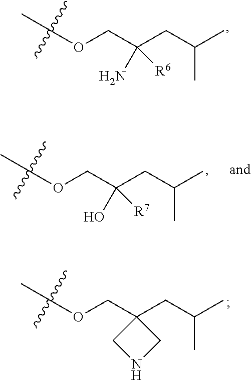
R⁶ is selected from hydrogen, ethyl, fluoromethyl, difluoromethyl, methyl, and trifluoromethyl; and
R⁷ is methyl.

2. The method of claim 1, wherein the AAK1 inhibitor is (S)-1-((2′,6-bis(difluoromethyl)-[2,4′-bipyridin]-5-yl) oxy)-2,4-dimethylpentan-2-amine: 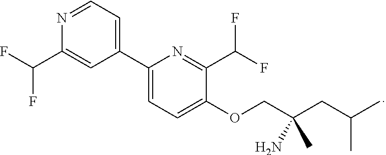 [SMILES: CC(C)C[C@](C)(N)COc1ccc(-c2ccnc(C(F)F)c2)nc1C(F)F]
or a pharmaceutically acceptable salt thereof.

3. The method of claim 2, wherein the CIPN is chronic.

4. The method of claim 2, wherein the CIPN is acute.

5. The method of claim 2, wherein the patient is undergoing chemotherapy, which chemotherapy comprises administering to the patient a chemotherapeutic agent.

6. The method of claim 5, wherein the AAK1 inhibitor is administered to the patient before the administration of the chemotherapeutic agent.

7. The method of claim 5, wherein the AAK1 inhibitor and chemotherapeutic agent are administered concurrently to the patient.

8. The method of claim 5, wherein the AAK1 inhibitor is administered to the patient after the administration of the chemotherapeutic agent.

9. The method of claim 5, wherein the chemotherapeutic agent and the AAK1 inhibitor are administered to the patient by the same route of administration.

10. (canceled)

11. (canceled)

12. (canceled)

13. (canceled)

14. (canceled)

15. The method of claim 5, wherein the patient is undergoing chemotherapy for the treatment of cancer, wherein the cancer is colorectal cancer, neuroblastoma, biliary adenocarcinoma, leukemia, esophageal cancer, gastric cancer, neuroendocrine tumors, non-Hodgkin lymphoma, pancreatic cancer, ovarian cancer, or testicular cancer.

16. The method of claim 15, wherein the cancer is colorectal cancer.

17. The method of claim 5, wherein the chemotherapeutic agent is chelated platinum, a taxane, a proteosome inhibitor, a vinca alkaloid, or an immunomodulatory drug.

18. The method of claim 17, wherein the chemotherapeutic agent is 5-fluorouracil, capecitabine, cisplatin, cytarabine, docetaxel, doxorubicin, epirubicin, etoposide, fludarabine, gemcitabine, ifosfamide, irinotecan, leucovorin, oxaliplatin, paclitaxel, or rituximab.

19. The method of claim 17, wherein the chemotherapeutic agent is chelated platinum.

20-54. (canceled)
